(19) United States
(12) Patent Application Publication
(10) Pub. No.: US 2025/0115658 A1
(43) Pub. Date: Apr. 10, 2025

(54) ENGINEERED PD-1 VARIANTS AND METHODS OF USE THEREOF

(71) Applicant: FBD BIOLOGICS LIMITED, Kowloon (HK)

(72) Inventors: Zong Sean Juo, New Taipei City (TW); Jiin-Tarng Wang, Taichung City (TW)

(21) Appl. No.: 18/729,212

(22) PCT Filed: Dec. 16, 2022
(86) PCT No.: PCT/US22/53153

§ 371 Date: Jul. 16, 2024

Related U.S. Application Data
(60) Provisional application No. 63/305,031, filed on Jan. 31, 2022.

(51) Int. Cl. C07K 14/705; A61K 38/00; A61P 35/00; C12N 15/86

(52) U.S. Cl. CPC C07K 14/70596; A61P 35/00; C07K 14/70503; C12N 15/86; A61K 38/00; C07K 2319/30

(57) ABSTRACT

This disclosure relates to engineered PD-1 variants, and methods of use thereof.

### CROSS-REFERENCE TO RELATED APPLICATION

This disclosure claims priority to and benefit of U.S. Provisional Patent Application Ser. No. 63/305,031, filed Jan. 31, 2022, which is incorporated herein by reference in its entirety.

### SEQUENCE LISTING

This application contains a Sequence Listing that has been submitted electronically as an XML file named 52246-0006WO1_SL ST26.xml. The XML file, created on Dec. 15, 2022, is 44,390 bytes in size. The material in the XML file is hereby incorporated by reference in its entirety.

### TECHNICAL FIELD

This disclosure relates to engineered PD-1 variants, and methods of use thereof.

### BACKGROUND

Programmed Cell Death Protein 1 (PD-1), also known as cluster of differentiation 279 (CD279), is a protein on the surface of T and B cells that has a role in regulating the immune system's response to the cells of the human body by down-regulating the immune system and promoting self-tolerance by suppressing T cell inflammatory activity.

The binding between PD-1 and PD-L1 is relatively weak (with a KD in the micro-molar range). To better target tumor cells expressing PD-L1, there is a need to develop a modified PD-1 variant with enhanced binding affinity to PD-L1.

### SUMMARY

This disclosure relates to engineered PD-1 variants, and methods of use thereof.

In one aspect, the disclosure is related to an engineered polypeptide comprising a PD-1 extracellular region and a PD-L1 surface interaction sequence with at least 5 amino acids, wherein the PD-L1 surface interaction sequence comprises two or more positively charged amino acids.

In some embodiments, the positively charged amino acids are selected from the group consisting of: histidine, arginine and lysine.

In some embodiments, the PD-L1 surface interaction sequence is positively charged.

In some embodiments, the engineered polypeptide has a higher binding affinity to PD-L1 relative to the PD-1 extracellular region without the PD-L1 surface interaction sequence.

In some embodiments, the off rate between the engineered polypeptide and PD-L1 is lower than the off rate between the PD-1 extracellular region without the PD-L1 surface interaction sequence and PD-L1.

In some embodiments, the PD-1 extracellular region comprises an amino acid sequence that is at least 80% identical to SEQ ID NO: 16.

In some embodiments, the PD-L1 surface interaction sequence is at the N-terminus or C-terminus of the engineered polypeptide.

In some embodiments, the PD-L1 surface interaction sequence is at the N-terminus of the engineered polypeptide

In some embodiments, the PD-L1 surface interaction sequence comprise a sequence that is at least 80% identical to SHGHGGG, SHHGHGHGGGG, SHGHHGHGGGG or SHGHGHHGGGG.

In some embodiments, one or more of the amino acids that corresponds to Y34, S39 and A95 of SEQ ID NO: 16 is H.

In some embodiments, the amino acid that corresponds to Y34 of SEQ ID NO: 16 is H.

In some embodiments, the amino acid that corresponds to S39 of SEQ ID NO: 16 is H.

In some embodiments, the amino acid that corresponds to A95 of SEQ ID NO: 16 is H.

In some embodiments, the engineered polypeptide comprises one of the following:
(a) the amino acid that corresponds to Y34 of SEQ ID NO: 16 is H;
(b) the amino acid that corresponds to S39 of SEQ ID NO: 16 is H;
(c) the amino acid that corresponds to A95 of SEQ ID NO: 16 is H;
(d) the amino acids that correspond to Y34 and S39 of SEQ ID NO: 16 are H;
(e) the amino acids that correspond to Y34 and A95 of SEQ ID NO: 16 are H;
(f) the amino acids that correspond to S39 and A95 of SEQ ID NO: 16 are H;
(g) the amino acids that correspond to Y34, S39 and A95 of SEQ ID NO: 16 are

In some embodiments, the PD-L1 surface interaction sequence has 5-15 amino acids.

In one aspect, the disclosure is related to an engineered polypeptide comprising an amino acid sequence that is at least 80% identical to SEQ ID NO: 16, wherein one or more of the amino acids that corresponds to Y34, S39 and A95 of SEQ ID NO: 16 is H.

In some embodiments, the amino acid that corresponds to Y34 of SEQ ID NO: 16 is H.

In some embodiments, the amino acid that corresponds to S39 of SEQ ID NO: 16 is H.

In some embodiments, the amino acid that corresponds to A95 of SEQ ID NO: 16 is H.

In some embodiments, the engineered polypeptide comprises one of the following:
(a) the amino acid that corresponds to Y34 of SEQ ID NO: 16 is H;
(b) the amino acid that corresponds to S39 of SEQ ID NO: 16 is H;
(c) the amino acid that corresponds to A95 of SEQ ID NO: 16 is H;
(d) the amino acids that correspond to Y34 and S39 of SEQ ID NO: 16 are H;
(e) the amino acids that correspond to Y34 and A95 of SEQ ID NO: 16 are H;
(f) the amino acids that correspond to S39 and A95 of SEQ ID NO: 16 are H; or
(g) the amino acids that correspond to Y34, S39 and A95 of SEQ ID NO: 16 are H.

In some embodiments, The engineered polypeptide further comprises a PD-L1 surface interaction sequence with at least 5 amino acids, wherein the PD-L1 surface interaction sequence comprises two or more histidine residues.

In some embodiments, the PD-L1 surface interaction sequence is at the N-terminus of the engineered polypeptide.

In some embodiments, the PD-L1 surface interaction sequence is selected from the group consisting of: SHGHGGG, SHHGHGHGGGG, SHGHHGHGGGG and SHGHGHHGGGG.

In some embodiments, PD-L1 surface interaction sequence has 5-15 amino acids.

In some embodiments, the PD-L1 surface interaction sequence interacts with E58/E60/D61 of PD-L1.

In some embodiments, the engineered polypeptide comprises an amino acid sequence that is at least 80%, 85%, 90%, or 95% identical to any one of SEQ ID NO: 1-16.

In some embodiments, the engineered polypeptide further comprises a CH2 domain and a CH3 domain.

In some embodiments, the engineered polypeptide further comprises a hinge region.

In some embodiments, the CH2 domain is an IgG CH2 domain and the CH3 domain is an IgG CH3 domain.

In some embodiments, the engineered polypeptide comprises an amino acid sequence that is at least 80%, 85%, 90%, or 95% identical to any one of SEQ ID NO: 19-34.

In one aspect, the disclosure is related to a protein construct comprising the engineered polypeptide described herein.

In some embodiments, the protein construct comprises at least two engineered polypeptides.

In some embodiments, the at least two engineered polypeptides are identical.

In some embodiments, the at least two engineered polypeptides are different.

In some embodiments, the protein construct further comprises an Fc region.

In some embodiments, the Fc region is an IgG4 Fc region.

In one aspect, the disclosure is related to a protein construct comprising a first fusion polypeptide comprising the engineered polypeptide described herein, a first CH2 domain, and a first CH3 domain; a second fusion polypeptide comprising a second CH2 domain, and a second CH3 domain, wherein the first fusion polypeptide and the second fusion polypeptide associate with each other, forming a dimer.

In some embodiments, the second fusion polypeptide further comprises a second engineered polypeptide.

In one aspect, the disclosure is related to a pharmaceutical composition comprising the engineered polypeptide described herein or the protein construct described herein; and a pharmaceutically acceptable carrier.

In one aspect, the disclosure is related to a nucleic acid encoding the engineered polypeptide described herein or the protein construct described herein.

In one aspect, the disclosure is related to a vector comprising the nucleic acids described herein.

In one aspect, the disclosure is related to a cell comprising the nucleic acids described herein.

In some embodiments, the cell is a CHO cell.

In one aspect, the disclosure is related to a method of producing an engineered polypeptide or a protein construct comprising the engineered polypeptide, the method comprising
(a) culturing the cell described herein under conditions sufficient for the cell to produce the engineered polypeptide or the protein construct; and
(b) collecting the engineered polypeptide or the protein construct produced by the cell.

In one aspect, the disclosure is related to a method of treating a subject having cancer, the method comprising administering a therapeutically effective amount of a composition comprising the engineered polypeptide described herein or the protein construct described herein, to the subject.

In some embodiments, the cancer cells express PD-L1.

In some embodiments, the cancer is melanoma, Hodgkin lymphoma, bladder cancer, kidney cancer, breast cancer, lung cancer (e.g., non-small cell lung cancer), head and neck squamous cell cancer, liver cancer, esophageal squamous cell cancer, colorectal cancer, cutaneous squamous cell carcinoma, or merkel cell carcinoma.

In one aspect, the disclosure is related to a method of decreasing the rate of tumor growth, the method comprising contacting a tumor cell with an effective amount of a composition comprising the engineered polypeptide described herein or the protein construct described herein.

In one aspect, the disclosure is related to a method of killing a tumor cell, the method comprising contacting a tumor cell with an effective amount of a composition comprising the engineered polypeptide described herein or the protein construct described herein.

As used herein, the term “engineered PD-1 polypeptide”, “engineered peptide”, or “engineered PD-1 variant” refers to a polypeptide derived from a wildtype PD-1 polypeptide or a portion thereof (e.g., the extracellular region of PD-1) with one or more mutations (e.g., insertions, deletions, or substitutions). In some embodiments, the engineered PD-1 polypeptide comprises or consists of the extracellular region of PD-1. In some embodiments, the engineered PD-1 polypeptide comprises a modified PD-1 extracellular domain. In some embodiments, the engineered PD-1 polypeptide comprises one or more antibody constant region sequences.

As used herein, the term “protein construct” refers to a complex having one or more polypeptides. In some embodiments, the protein construct has two or more polypeptides, wherein the polypeptides can associate with each other, forming a dimer or a multimer.

As used herein, the term “cancer” refers to cells having the capacity for autonomous growth. Examples of such cells include cells having an abnormal state or condition characterized by rapidly proliferating cell growth. The term is meant to include cancerous growths, e.g., tumors; oncogenic processes, metastatic tissues, and malignantly transformed cells, tissues, or organs, irrespective of histopathologic type or stage of invasiveness. Also included are malignancies of the various organ systems, such as respiratory, cardiovascular, renal, reproductive, hematological, neurological, hepatic, gastrointestinal, and endocrine systems; as well as adenocarcinomas which include malignancies such as most colon cancers, renal-cell carcinoma, prostate cancer and/or testicular tumors, non-small cell carcinoma of the lung, and cancer of the small intestine. Cancer that is “naturally arising” includes any cancer that is not experimentally induced by implantation of cancer cells into a subject, and includes, for example, spontaneously arising cancer, cancer caused by exposure of a patient to a carcinogen(s), cancer resulting from insertion of a transgenic oncogene or knockout of a tumor suppressor gene, and cancer caused by infections, e.g., viral infections. The term “carcinoma” is art recognized and refers to malignancies of epithelial or endocrine tissues. The term also includes carcinosarcomas, which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation. The term “hematopoietic neoplastic disorders” includes diseases involving hyperplastic/neoplastic cells of hematopoietic origin. A hematopoietic neoplastic disorder can arise from myeloid, lymphoid or erythroid lineages, or precursor cells thereof. A hematologic cancer is a cancer that begins in blood-forming tissue, such as the bone marrow, or in the cells of the immune system. Examples of hematologic cancer include e.g., leukemia, lymphoma, and multiple myeloma etc.

As used herein, the terms “subject” and “patient” are used interchangeably throughout the specification and describe an animal, human or non-human, to whom treatment according to the methods of the present invention is provided. Veterinary and non-veterinary applications are contemplated in the present disclosure. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

As used herein, the terms “polypeptide,” “peptide,” and “protein” are used interchangeably to refer to polymers of amino acids of any length of at least two amino acids.

As used herein, the terms “polynucleotide,” “nucleic acid molecule,” and “nucleic acid sequence” are used interchangeably herein to refer to polymers of nucleotides of any length of at least two nucleotides, and include, without limitation, DNA, RNA, DNA/RNA hybrids, and modifications thereof.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 shows the 3D protein structure of the binding between human PD-1 and PD-L1. Three amino acids at the binding interface (A129, Y68 and S73) are identified.

FIG. 2 lists certain amino acid sequences discussed in this disclosure.

FIG. 3 is a table showing the production and CMC analysis for PD1 mutants with IgG4 Fc.

FIGS. 4A-4B are tables showing the binding affinity results of hPD-1-Fc proteins to PDL1-ECD-His by an Octet® system at different pH.

FIG. 5 shows a schematic structure of hPD-1-Fc proteins. Two PD-1 extracellular domains are connected to the N-terminus of human IgG4 Fc regions (e.g., through hinge).

FIGS. 6A-6D show whole cell binding results of hPD-1-Fc proteins to PD-L1 transfected CHO—S cells at different pH.

FIGS. 7A-7B show in vitro binding results of hPD-1-Fc proteins to PD-L2 as measured by ELISA.

FIGS. 8A-8B show in vitro binding results of hPD-1-Fc proteins to Cyno PD-L1 and mouse PD-L1 as measured by ELISA.

FIGS. 9A-9D show whole cell blocking results of hPD-1-Fc proteins to PD-L1 transfected CHO—S cells at different pH.

FIGS. 10A-10F show in vitro function of PD1-Fc-mts in MLR (donor 023), as measured by proliferation, IL-2 secretion and IFN-γ secretion.

FIGS. 11A-11C show in vitro function of PD1-Fc-mts in MLR (donor 015), as measured by proliferation, IL-2 secretion and IFN-γ secretion.

FIGS. 12A-12F show in vitro function of PD1-Fc-mts in MLR (donor 025), as measured by proliferation, IL-2 secretion and IFN-γ secretion.

FIGS. 13A-13F show in vitro function of PD1-Fc-mts in MLR (donor 046), as measured by proliferation, IL-2 secretion and IFN-γ secretion.

FIG. 14 shows T cell response in MLR assay in different donors. The + sign represents the enhancement fold compared to T+DC group: (0˜1):−; (1˜2): +; (2˜3): ++; (3˜4): +++; (4˜10): ++++.

FIG. 15 lists certain amino acid sequences discussed in the disclosure.

### DETAILED DESCRIPTION

The present disclosure provides engineered PD-1 variants. These engineered PD-1 variants can be used to target PD-L1/PD-1 pathway, whereas the interaction of engineered PD-1 variants and PD-L1 are carefully modulated. In one aspect, given the acidic nature of the tumor micro-environment, the disclosure also provides a modified PD-1 variant that has good binding affinity to PD-L1 under lower pH.

### Engineered PD-1 Variants

This disclosure relates to engineered PD-1 (Programmed Cell Death Protein 1; also known as CD279) variants, and methods of use thereof.

PD-1 (programmed death-1) is an immune checkpoint and guards against autoimmunity through a dual mechanism of promoting apoptosis (programmed cell death) in antigen-specific T-cells in lymph nodes while simultaneously reducing apoptosis in regulatory T cells (anti-inflammatory, suppressive T cells).

PD-1 is mainly expressed on the surfaces of T cells and primary B cells; two ligands of PD-1 (PD-L1 and PD-L2) are widely expressed in antigen-presenting cells (APCs). The interaction of PD-1 with its ligands plays an important role in the negative regulation of the immune response. Inhibition the binding between PD-1 and its ligand can make the tumor cells exposed to the killing effect of the immune system, and thus can reach the effect of killing tumor tissues and treating cancers.

PD-L1 is expressed on the neoplastic cells of many different cancers. By binding to PD-1 on T-cells leading to its inhibition, PD-L1 expression is a major mechanism by which tumor cells can evade immune attack. PD-L1 over-expression may conceptually be due to 2 mechanisms, intrinsic and adaptive. Intrinsic expression of PD-L1 on cancer cells is related to cellular/genetic aberrations in these neoplastic cells. Activation of cellular signaling including the AKT and STAT pathways results in increased PD-L1 expression. In primary mediastinal B-cell lymphomas, gene fusion of the MHC class II transactivator (CIITA) with PD-L1 or PD-L2 occurs, resulting in over expression of these proteins. Amplification of chromosome 9p23-24, where PD-L1 and PD-L2 are located, leads to increased expression of both proteins in classical Hodgkin lymphoma. Adaptive mechanisms are related to induction of PD-L1 expression in the tumor microenvironment. PD-L1 can be induced on neoplastic cells in response to interferon γ. In microsatellite instability colon cancer, PD-L1 is mainly expressed on myeloid cells in the tumors, which then suppress cytotoxic T-cell function.

The use of PD-1 blockade to enhance anti-tumor immunity originated from observations in chronic infection models, where preventing PD-1 interactions reversed T-cell exhaustion. Similarly, blockade of PD-1 prevents T-cell PD-1/tumor cell PD-L1 or T-cell PD-1/tumor cell PD-L2 interaction, leading to restoration of T-cell mediated anti-tumor immunity.

A detailed description of PD-1, and the use of anti-PD-1 antibodies to treat cancers are described, e.g., in Topalian, Suzanne L., et al. “Safety, activity, and immune correlates of anti-PD-1 antibody in cancer.” New England Journal of Medicine 366.26 (2012): 2443-2454; Hirano, Fumiya, et al. “Blockade of B7-H1 and PD-1 by monoclonal antibodies potentiates cancer therapeutic immunity.” Cancer research 65.3 (2005): 1089-1096; Raedler, Lisa A. “Keytruda (pembrolizumab): first PD-1 inhibitor approved for previously treated unresectable or metastatic melanoma.” American health & drug benefits 8.Spec Feature (2015): 96; Kwok, Gerry, et al. “Pembrolizumab (Keytruda).” (2016): 2777-2789; US20170247454; U.S. Pat. Nos. 9,834,606 B; and 8,728,474; each of which is incorporated by reference in its entirety.

According to UniProt identifier Q15116, the extracellular region of human PD-1 corresponds to amino acids 24-170 of SEQ ID NO: 17, the transmembrane region of human PD-1 corresponds to amino acids 171-191 of SEQ ID NO: 17, and the cytoplasmic region of human PD-1 corresponds to amino acids 192-288 of SEQ ID NO: 17. The PD-1 extracellular region also has an IgV domain, which corresponds to amino acids 35-145 of the human PD-1 protein (SEQ ID NO: 17; NP_005009.2). The signal peptide corresponds to amino acids 1-23 of SEQ ID NO: 17.

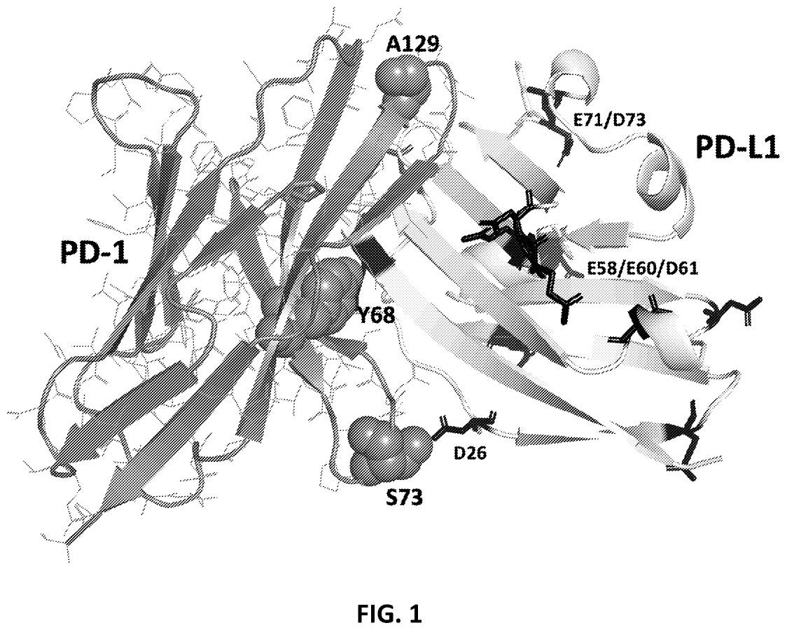

Based on the structure of human PD-1 in complex with PD-L1, the interacting residues with PD-L1 are determined. The analysis shows multiple interacting residues. In FIG. 1, three PD-1 amino acid residues (A129, Y68 and S73) that may interact with Glu or Asp residues in PD-L1 (E71/D73, D122 and D26) are identified. In addition, additional histidine residues at the N-terminus of PD-1 will be in close proximity of E58/E60/D61 of PD-L1. These regions are the targets for mutations. Thus, in some embodiments, the engineered PD-1 polypeptide comprises or consists of a PD-1 extracellular domain. As used herein, the “PD-1 extracellular domain” refers to the entire or a portion of the extracellular region of PD-1 or the variant thereof, wherein the portion of the extracellular region can bind to PD-L1. The PD-1 extracellular domain can have one or more protein domains that can fold independently and form self-stabilizing structures. In some embodiments, the PD-1 extracellular domain comprises or consists of the IgV domain. In some embodiments, the PD-1 extracellular domain does not include the signal peptide.

In some embodiments, the PD-1 extracellular domain described herein includes an amino acid sequence that is at least 80%, 85%, 90%, 95%, 96%, 97%, 98%, 99%, or 100% identical to amino acids 26-170 or 35-170 of human PD-1 protein (NCBI Accession No.: NP_005009.2; SEQ ID NO: 17). In some embodiments, the PD-1 extracellular domain described herein includes an amino acid sequence that is at least 80%, 85%, 90%, 95%, 96%, 97%, 98%, 99%, or 100% identical to SEQ ID NO: 15 or 16. In some embodiments, the PD-1 extracellular domain described herein includes the IgV domain of human PD-1 protein. In some embodiments, the PD-1 extracellular domain described herein includes at least or about 1, 2, 3, 4, 5 6, 7, 8, 9, 10, or more than 10 mutations

In some embodiments, the engineered PD-1 polypeptide comprises one or more amino acid mutations at A129, Y68, S73 (based on sequence of SEQ ID NO: 17) and/or its N-terminus. In some embodiments, the engineered PD-1 polypeptide comprises or consists of one or more amino acid mutations selected from A129H, Y68H and S73H. In some embodiments, the engineered PD-1 polypeptide comprises or consists of one or more additional N-terminal histidine residues.

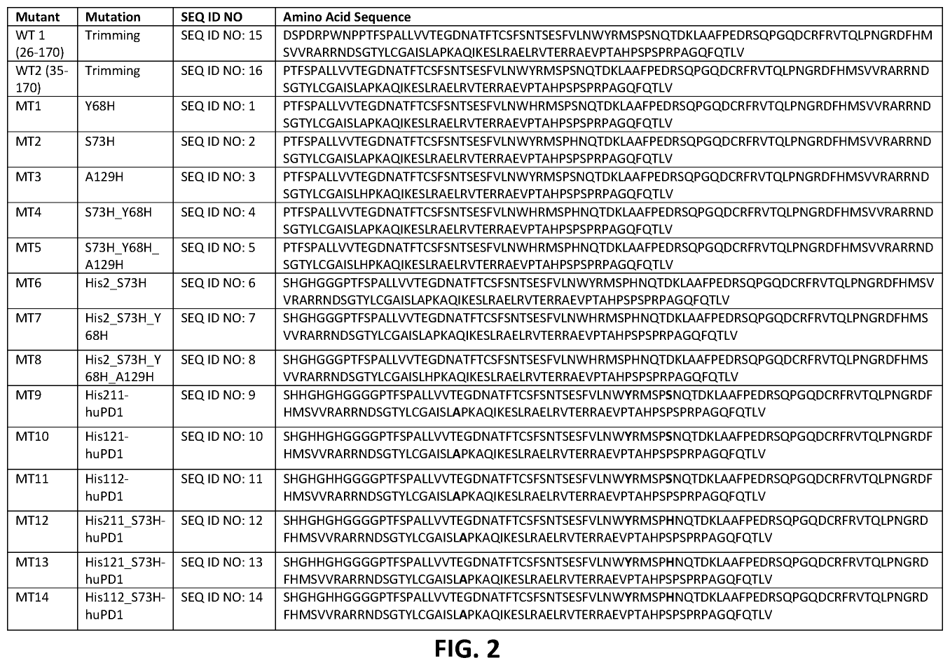

Based on the structural analysis, PD-1 mutants were constructed by making amino acid mutations selected from A129H, Y68H and S73H, and by adding additional N-terminal histidine residues. The sequences of the PD-1 mutants are shown in FIG. 2. Thus, in one aspect, the engineered PD-1 polypeptide comprises or consists of an amino acid sequence that is at least 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 1, 2, 3, 4, 5, 6, 7, 8, 9, 10, 11, 12, 13, or 14.

In some embodiments, the engineered PD-1 variants can have at least or about 1 (e.g., at least or about 2, 3, 4, 5, 6, 7, 8, 9, 10, 20, 30, 40) amino acid insertions, deletions, or substitutions as compared to any one of SEQ ID NO: 1-16.

In some embodiments, the engineered PD-1 polypeptide has one or more of the following mutations: A129H, Y68H and S73H.

In some embodiments, the engineered PD-1 polypeptide comprises or consists of one or more of the following mutations: trimming the wildtype PD-1 sequence (SEQ ID NO: 17) down to amino acids 26-170 (SEQ ID NO: 15); trimming the wildtype PD-1 sequence down to amino acids 35-170 (SEQ ID NO: 16); trimming the wildtype PD-1 sequence down to amino acids 35-170 and adding one of the below PD-L1 surface interaction sequences at the N-terminus: SHGHGGG (His2), SHHGHGHGGGG (His211), SHGHHGHGGGG (His121) and SHGHGHHGGGG (His112).

In some embodiments, the engineered PD-1 polypeptide comprises or consists of an amino acid sequence that is at least 80%, 85%, 90%, or 95% identical to any one of SEQ ID NOs: 1-16.

In some embodiments, the engineered PD-1 polypeptide comprises or consists of one or more of the following mutations:
(a) the amino acid that corresponds to Y68 of SEQ ID NO: 17 is H;
(b) the amino acid that corresponds to S73 of SEQ ID NO: 17 is H;
(c) the amino acid that corresponds to A129 of SEQ ID NO: 17 is H;
(d) trimming the wildtype PD-1 sequence (SEQ ID NO: 17) down to amino acids 35-170 (SEQ ID NO: 16).

In some embodiments, the engineered PD-1 polypeptide comprises or consists of an amino acid sequence that is at least 80%, 85%, 90%, or 95% identical to any one of SEQ ID

NOs: 1-5.

In some embodiments, the engineered PD-1 polypeptide comprises or consists of one or more of the following mutations:
(a) the amino acid that corresponds to Y68 of SEQ ID NO: 17 is H;
(b) the amino acid that corresponds to S73 of SEQ ID NO: 17 is H;
(c) the amino acid that corresponds to A129 of SEQ ID NO: 17 is H;
(d) trimming the wildtype PD-1 sequence (SEQ ID NO: 17) down to amino acids 35-170 (SEQ ID NO: 16);
(e) adding one of the below PD-L1 surface interaction sequences at the N-terminus: SHGHGGG (His2), SHHGHGHGGGG (His211), SHGHHGHGGGG (His121) and SHGHGHHGGGG (His112).

In some embodiments, the engineered PD-1 polypeptide comprises or consists of an amino acid sequence that is at least 80%, 85%, 90%, or 95% identical to any one of SEQ ID NOs: 6-14.

In some embodiments, the engineered PD-1 polypeptide comprises or consists of one or more of the following mutations:
(a) the amino acid that corresponds to Y68 of SEQ ID NO: 17 is H;
(b) the amino acid that corresponds to S73 of SEQ ID NO: 17 is H;
(c) the amino acid that corresponds to A129 of SEQ ID NO: 17 is H;
(d) trimming the wildtype PD-1 sequence (SEQ ID NO: 17) down to amino acids 35-170 (SEQ ID NO: 16);
(e) adding the PD-L1 surface interaction sequence SHGHGGG (His2) at the N-terminus.

In some embodiments, the engineered PD-1 polypeptide comprises or consists of an amino acid sequence that is at least 80%, 85%, 90%, or 95% identical to any one of SEQ ID NOs: 6-8.

In some embodiments, the engineered PD-1 polypeptide comprises or consists of one or more of the following mutations:
(a) the amino acid that corresponds to Y68 of SEQ ID NO: 17 is H;
(b) the amino acid that corresponds to S73 of SEQ ID NO: 17 is H;
(c) the amino acid that corresponds to A129 of SEQ ID NO: 17 is H;
(d) trimming the wildtype PD-1 sequence (SEQ ID NO: 17) down to amino acids 35-170 (SEQ ID NO: 16);
(e) adding the PD-L1 surface interaction sequence SHHGHGHGGGG (His211) at the N-terminus.

In some embodiments, the engineered PD-1 polypeptide comprises or consists of an amino acid sequence that is at least 80%, 85%, 90%, or 95% identical to any one of SEQ ID NOs: 9 and 12.

In some embodiments, the engineered PD-1 polypeptide comprises or consists of one or more of the following mutations:
(a) the amino acid that corresponds to Y68 of SEQ ID NO: 17 is H;
(b) the amino acid that corresponds to S73 of SEQ ID NO: 17 is H;
(c) the amino acid that corresponds to A129 of SEQ ID NO: 17 is H;
(d) trimming the wildtype PD-1 sequence (SEQ ID NO: 17) down to amino acids 35-170 (SEQ ID NO: 16);
(e) adding the PD-L1 surface interaction sequence SHGHHGHGGGG (His121) at the N-terminus.

In some embodiments, the engineered PD-1 polypeptide comprises or consists of an amino acid sequence that is at least 80%, 85%, 90%, or 95% identical to any one of SEQ ID NOs: 10 and 13.

In some embodiments, the engineered PD-1 polypeptide comprises or consists of one or more of the following mutations:
(a) the amino acid that corresponds to Y68 of SEQ ID NO: 17 is H;
(b) the amino acid that corresponds to S73 of SEQ ID NO: 17 is H;
(c) the amino acid that corresponds to A129 of SEQ ID NO: 17 is H;
(d) trimming the wildtype PD-1 sequence (SEQ ID NO: 17) down to amino acids 35-170 (SEQ ID NO: 16);
(e) adding the PD-L1 surface interaction sequence SHGHGHHGGGG (His112) at the N-terminus.

In some embodiments, the engineered PD-1 polypeptide comprises or consists of an amino acid sequence that is at least 80%, 85%, 90%, or 95% identical to any one of SEQ ID

NOs: 11 and 14.

In one aspect, the engineered polypeptide comprises an amino acid sequence that is at least 80% identical to SEQ ID NO: 16, wherein one or more of the amino acids that corresponds to Y34, S39 and A95 of SEQ ID NO: 16 is H.

In some embodiments, the engineered polypeptide comprises or consists of one of the following mutations:
(a) the amino acid that corresponds to Y34 of SEQ ID NO: 16 is H;
(b) the amino acid that corresponds to S39 of SEQ ID NO: 16 is H;
(c) the amino acid that corresponds to A95 of SEQ ID NO: 16 is H;
(d) the amino acids that correspond to Y34 and S39 of SEQ ID NO: 16 are H;
(e) the amino acids that correspond to Y34 and A95 of SEQ ID NO: 16 are H;
(f) the amino acids that correspond to S39 and A95 of SEQ ID NO: 16 are H; or
(g) the amino acids that correspond to Y34, S39 and A95 of SEQ ID NO: 16 are H.

In some embodiments, the engineered polypeptide comprises a PD-L1 surface interaction sequence comprising about or at least 2, 3, 4, 5, 6, 7, 8, 9, 10, 11, 12, 13, 14, 15, 20, or 25 amino acids, wherein the PD-L1 surface interaction sequence comprises two or more histidine residues. In some embodiments, the PD-L1 surface interaction sequence comprises or consists of about or at least 5, 6, 7, 8, 9, 10, 11, 12, 13, 14, 15, 16, 17, 18, 19, 20, 25, 30, 40, or 50 amino acids. In some embodiments, the PD-L1 surface interaction sequence comprises or consists of at most 5, 6, 7, 8, 9, 10, 11, 12, 13, 14, 15, 16, 17, 18, 19, 20, 25, 30, 40, or 50 amino acids. In some embodiments, the PD-L1 surface interaction sequence comprises or consists of 5-15, 5-20, 5-30, 5-40, 10-15, 10-20, 10-30, 10-40, 15-20, 15-30, or 15-40 amino acids. In some embodiments, the PD-L1 surface interaction sequence comprises or consists of 5-15 amino acids.

In some embodiments, the PD-L1 surface interaction sequence comprises or consists of about or at least 2, 3, 4, 5, or 6 histidine residues. In some embodiments, the PD-L1 surface interaction sequence comprises or consists of at most 2, 3, 4, 5, or 6 histidine residues. In some embodiments, the PD-L1 surface interaction sequence comprises or consists of 2-3, 2-4, 2-5 or 2-6 histidine residues. In some embodiments, the PD-L1 surface interaction sequence comprises or consists of 2-4 histidine residues.

In some embodiments, the PD-L1 surface interaction sequence comprises or consists of about or at least 2, 3, 4, 5, 6, 7, 8, 9, or 10 positively charged amino acid residues (e.g., histidine, lysine or arginine). In some embodiments, the PD-L1 surface interaction sequence comprises or consists of at most 2, 3, 4, 5, 6, 7, 8, 9, or 10 positively charged amino acid residues (e.g., histidine, lysine or arginine). In some embodiments, the PD-L1 surface interaction sequence comprises or consists of 2-3, 2-4, 2-5, 2-6, 2-10, 3-10, or 5-10 positively charged amino acid residues (e.g., histidine, lysine or arginine). In some embodiments, the PD-L1 surface interaction sequence comprises or consists of 2-4 positive amino acid residues (e.g., histidine, lysine or arginine).

In some embodiments, the PD-L1 surface interaction sequence is at the N-terminus of the engineered polypeptide. In some embodiments, the PD-L1 surface interaction sequence is at the C-terminus of the engineered polypeptide.

In some embodiments, the PD-L1 surface interaction sequence is selected from the group consisting of: SHGHGGG, SHHGHGHGGGG, SHGHHGHGGGG and SHGHGHHGGGG.

In some embodiments, the engineered PD-1 polypeptide comprises or consists of one or more of the following mutations:
(a) the amino acid that corresponds to Y34 of SEQ ID NO: 16 is H;
(b) the amino acid that corresponds to S39 of SEQ ID NO: 16 is H;
(c) the amino acid that corresponds to A195 of SEQ ID NO: 16 is H;

In some embodiments, the engineered PD-1 polypeptide comprises or consists of an amino acid sequence that is at least 80%, 85%, 90%, or 95% identical to any one of SEQ ID NOs: 1-5.

In some embodiments, the engineered PD-1 polypeptide comprises or consists of one or more of the following mutations:
(a) the amino acid that corresponds to Y34 of SEQ ID NO: 16 is H;
(b) the amino acid that corresponds to S39 of SEQ ID NO: 16 is H;
(c) the amino acid that corresponds to A195 of SEQ ID NO: 16 is H;
(d) adding one of the below PD-L1 surface interaction sequences at the N-terminus: SHGHGGG (His2), SHHGHGHGGGG (His211), SHGHHGHGGGG (His121) and SHGHGHHGGGG (His112).

In some embodiments, the engineered PD-1 polypeptide comprises or consists of an amino acid sequence that is at least 80%, 85%, 90%, or 95% identical to any one of SEQ ID NOs: 6-14.

In some embodiments, the engineered PD-1 polypeptide comprises or consists of one or more of the following mutations:
(a) the amino acid that corresponds to Y34 of SEQ ID NO: 16 is H;
(b) the amino acid that corresponds to S39 of SEQ ID NO: 16 is H;
(c) the amino acid that corresponds to A195 of SEQ ID NO: 16 is H;
(d) adding the PD-L1 surface interaction sequence SHGHGGG (His2) at the N-terminus.

In some embodiments, the engineered PD-1 polypeptide comprises or consists of an amino acid sequence that is at least 80%, 85%, 90%, or 95% identical to any one of SEQ ID NOs: 6-8.

In some embodiments, the engineered PD-1 polypeptide comprises or consists of one or more of the following mutations:
(a) the amino acid that corresponds to Y34 of SEQ ID NO: 16 is H;
(b) the amino acid that corresponds to S39 of SEQ ID NO: 16 is H;
(c) the amino acid that corresponds to A195 of SEQ ID NO: 16 is H;
(d) adding the PD-L1 surface interaction sequence SHHGHGHGGGG (His211) at the N-terminus.

In some embodiments, the engineered PD-1 polypeptide comprises or consists of an amino acid sequence that is at least 80%, 85%, 90%, or 95% identical to any one of SEQ ID NOs: 9 and 12.

In some embodiments, the engineered PD-1 polypeptide comprises or consists of one or more of the following mutations:
(a) the amino acid that corresponds to Y34 of SEQ ID NO: 16 is H;
(b) the amino acid that corresponds to S39 of SEQ ID NO: 16 is H;
(c) the amino acid that corresponds to A195 of SEQ ID NO: 16 is H;
(d) adding the PD-L1 surface interaction sequence SHGHHGHGGGG (His121) at the N-terminus.

In some embodiments, the engineered PD-1 polypeptide comprises or consists of an amino acid sequence that is at least 80%, 85%, 90%, or 95% identical to any one of SEQ ID NOs: 10 and 13.

In some embodiments, the engineered PD-1 polypeptide comprises or consists of one or more of the following mutations:
(a) the amino acid that corresponds to Y34 of SEQ ID NO: 16 is H;
(b) the amino acid that corresponds to S39 of SEQ ID NO: 16 is H;
(c) the amino acid that corresponds to A195 of SEQ ID NO: 16 is H;
(d) adding the PD-L1 surface interaction sequence SHGHGHHGGGG (His112) at the N-terminus.

In some embodiments, the engineered PD-1 polypeptide comprises or consists of an amino acid sequence that is at least 80%, 85%, 90%, or 95% identical to any one of SEQ ID NOs: 11 and 14.

The engineered PD-1 polypeptide can have additional modifications. In some embodiments, the engineered PD-1 polypeptide can have a CH2 domain and/or a CH3 domain. In some embodiments, the engineered PD-1 polypeptide can be linked to the N-terminus of the CH2 domain (e.g., through an optional hinge region or a GS linker). In some embodiments, the engineered PD-1 polypeptide can be linked to the C-terminus of the CH3 domain (e.g., through an optional GS linker). In some embodiments, the hinge region is an

IgG hinge region (e.g., IgG4 hinge region). In some embodiments, the CH2 domain is an IgG CH2 domain (e.g., IgG4 CH2 domain). In some embodiments, the CH3 domain is an IgG CH3 domain (e.g., IgG4 CH3 domain). In some embodiments, the hinge region, the CH2 domain, the CH3 domain have a sequence that is at least 80%, 85%, 90%, 95%, 100% identical to SEQ ID NO: 35.

In some embodiments, the engineered PD-1 polypeptide comprises or consists of an amino acid sequence that is at least 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 19, 20, 21, 22, 23, 24, 25, 26, 27, 28, 29, 30, 31, 32, 33 or 34.

### PD-1 Protein Constructs

The disclosure provides engineered PD-1 protein constructs that can specifically bind to PD-L1. The engineered PD-1 variant can be an engineered PD-1 protein construct. In some embodiments, these protein constructs can block PD-L1/PD-1 signaling pathway thus increase immune response. In some embodiments, these protein constructs can initiate phagocytosis.

In some embodiments, the engineered PD-1 protein constructs can comprise any engineered PD-1 variant as described herein. In some embodiments, the engineered PD-1 protein constructs can have a sequence that is at least 80%, 81%, 82%, 83%, 84%, 85%, 86%, 87%, 88%, 89% 90%, 91%, 92%, 93%, 94%, 95%, 96%, 97%, 98%, 99% or 100% identical to any sequence of SEQ ID NO: 1-14. In some embodiments, the engineered PD-1 protein constructs can comprise or consists of a sequence that is at least 80%, 81%, 82%, 83%, 84%, 85%, 86%, 87%, 88%, 89% 90%, 91%, 92%, 93%, 94%, 95%, 96%, 97%, 98%, 99% or 100% identical to any sequence of SEQ ID NO: 19-34.

The disclosure also provides nucleic acid comprising a polynucleotide encoding a polypeptide comprising a sequence that is at least 80%, 81%, 82%, 83%, 84%, 85%, 86%, 87%, 88%, 89% 90%, 91%, 92%, 93%, 94%, 95%, 96%, 97%, 98%, 99% or 100% identical to any sequence of SEQ ID NO: 1-14 or SEQ ID NO: 19-34.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90%, 95%, or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For example, the comparison of sequences and determination of percent identity between two sequences can be accomplished using a Blossum 62 scoring matrix with a gap penalty of 12, a gap extend penalty of 4, and a frameshift gap penalty of 5.

The engineered PD-1 protein constructs can further comprises an Fc region of an antibody. These antibodies can be of any type (e.g., IgG, IgE, IgM, IgD, IgA, and IgY), class or subclass (e.g., IgG1, IgG2, IgG3, IgG4, IgA1, IgA2, IgE1, IgE2). In some embodiments, the Fc region is derived from human IgG (e.g., IgG1, IgG2, IgG3, or IgG4). In some embodiments, the Fc region is an IgG4 Fc region (e.g., human IgG4 Fc region).

In some embodiments, the engineered PD-1 variant is linked to the Fc region through an antibody hinge region (e.g., IgG, IgE hinge region). In addition, the Fc region can be modified to provide desired effector functions or serum half-life.

The engineered PD-1 variants and/or protein constructs described herein can block the binding between PD-L1 and endogenous PD-1 that are expressed on immune cells. In some embodiments, by binding to PD-L1, the engineered PD-1 variants and/or protein constructs can inhibit the binding of PD-L1 (e.g., that is expressed on tumor cells) to endogenous PD-1 that is expressed on immune cells (e.g., myeloid cells, macrophages and dendritic cells), thereby blocking PD-L1/PD-1 pathway, upregulating immune response, and promoting phagocytosis.

In some embodiments, the engineered PD-1 variants and/or protein constructs as described herein can increase immune response, activity or number of immune cells (e.g., myeloid cells, macrophages, dendritic cells, antigen presenting cells) by at least 10%, 20%, 30%, 40%, 50%, 60%, 70%, 80%, 90%, 100%, 2 folds, 3 folds, 5 folds, 10 folds, or 20 folds.

In some implementations, the engineered PD-1 variants and/or protein constructs can bind to PD-L1 (e.g., human PD-L1, monkey PD-L1 (e.g., cynomolgus monkey (Macaca fascicularis), mouse PD-L1) with a dissociation rate (koff) of less than 0.1 s⁻¹, less than 0.01 s⁻¹, less than 0.001 s⁻¹, less than 0.0001 s⁻¹, or less than 0.00001 s⁻¹. In some embodiments, the dissociation rate (koff) is greater than 0.01 s⁻¹, greater than 0.001 s⁻¹, greater than 0.0001 s⁻¹, greater than 0.00001 s⁻¹, or greater than 0.000001 s⁻¹.

In some embodiments, kinetic association rates (kon) is greater than 1×10²/Ms, greater than 1×10³/Ms, greater than 1×10⁴/Ms, greater than 1×10⁵/Ms, or greater than 1×10⁶/Ms. In some embodiments, kinetic association rates (kon) is less than 1×10⁵/Ms, less than 1×10⁶/Ms, or less than 1×10⁷/Ms.

Affinities can be deduced from the quotient of the kinetic rate constants (KD=koff/kon). In some embodiments, KD is less than 1×10⁻⁶M, less than 1×10⁻⁷M, less than 1×10⁻⁸ M, less than 1×10⁻⁹ M, or less than 1×10⁻¹⁰ M. In some embodiments, the KD is less than 300 nM, 200 nM, 100 nM, 50 nM, 30 nM, 20 nM, 15 nM, 10 nM, 9 nM, 8 nM, 7 nM, 6 nM, 5 nM, 4 nM, 3 nM, 2 nM, 1 nM, 900 μM, 800 μM, 700 μM, 600 μM, 500 μM, 400 μM, 300 μM, 200 μM, 100 μM, 90 μM, 80 μM, 70 μM, 60 μM, 50 μM, 40 μM, 30 μM, 20 μM, or 10 μM. In some embodiments, KD is greater than 1×10⁻⁷M, greater than 1×10⁻⁸ M, greater than 1×10⁻⁹ M, greater than 1×10⁻¹⁰ M, greater than 1×10⁻¹¹ M, or greater than 1×10⁻¹² M.

General techniques for measuring the affinity include, e.g., ELISA, RIA, and surface plasmon resonance (SPR). In some embodiments, the engineered PD-1 variants and/or protein constructs can bind to monkey PD-L1, and/or mouse PD-L1. In some embodiments, the engineered PD-1 variants and/or protein constructs cannot bind to monkey PD-L1, and/or mouse PD-L1.

In some embodiments, thermal stabilities are determined. The engineered PD-1 variants and/or protein constructs as described herein can have a Tm greater than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C. In some embodiments, Tm is less than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C.

In some embodiments, the engineered PD-1 variants and/or protein constructs as described herein has a tumor growth inhibition percentage (TGI %) that is greater than 10%, 20%, 30%, 40%, 50%, 60%, 70%, 80%, 90%, 100%, 110%, 120%, 130%, 140%, 150%, 160%, 170%, 180%, 190%, or 200%. In some embodiments, the engineered PD-1 variants and/or protein constructs as described herein has a tumor growth inhibition percentage that is less than 60%, 70%, 80%, 90%, 100%, 110%, 120%, 130%, 140%, 150%, 160%, 170%, 180%, 190%, or 200%. The TGI % can be determined, e.g., at 3, 4, 5, 6, 7, 8, 9, 10, 11, 12, 13, 14, 15, 16, 17, 18, 19, 20, 21, 22, 23, 24, 25, 26, 27, 28, 29, or 30 days after the treatment starts, or 1, 2, 3, 4, 5, 6, 7, 8, 9, 10, 11, or 12 months after the treatment starts. As used herein, the tumor growth inhibition percentage (TG1%) is calculated using the following formula:

$$TGI ( \% ) = [ 1 - ( T i - T 0 ) / ( Vi - V 0 ) ] \times 100$$

Ti is the average tumor volume in the treatment group on day i. T0 is the average tumor volume in the treatment group on day zero. Vi is the average tumor volume in the control group on day i. V0 is the average tumor volume in the control group on day zero.

In some embodiments, the tumor inhibitory effects of the engineered PD-1 variants and/or protein constructs as described herein are comparable to an anti-PD-L1 reference antibody, e.g., Atezolizumab, Avelumab, Durvalumab, or MPDL3280A, or an anti-PD-1 antibody. In some embodiments, the tumor inhibitory effects of the engineered PD-1 variants and/or protein constructs as described herein are at least 10%, 20%, 30%, 40%, 50%, 60%, 70%, 80%, 90%, 1 fold, 2 folds, or 5 folds more than an anti-PD-L1 reference antibody.

In some embodiments, the protein constructs as described herein have a functional Fc region. In some embodiments, the Fc region is human IgG1, human IgG2, human IgG3, or human IgG4. In some embodiments, effector function of a functional Fc region is antibody-dependent cell-mediated cytotoxicity (ADCC). In some embodiments, effector function of a functional Fc region is phagocytosis. In some embodiments, effector function of a functional Fc region is ADCC and phagocytosis. In some embodiments, the protein constructs as described herein have an Fc region without effector function. In some embodiments, the Fc is a human IgG4 Fc. In some embodiments, the Fc does not have a functional Fc region. For example, the Fc region has LALA mutations (L234A and L235A mutations in EU numbering), or LALA-PG mutations (L234A, L235A, P329G mutations in EU numbering).

Some other modifications to the Fc region can be made. For example, a cysteine residue(s) can be introduced into the Fc region, thereby allowing interchain disulfide bond formation in this region. The homodimeric fusion protein thus generated may have any increased half-life in vitro and/or in vivo.

In some embodiments, the IgG4 has S228P mutation (EU numbering). The S228P mutation prevents in vivo and in vitro IgG4 Fab-arm exchange.

In some embodiments, Fc regions are provided having a carbohydrate structure that lacks fucose attached (directly or indirectly) to an Fc region. For example, the amount of fucose in such Fc region composition may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues; or position 314 in Kabat numbering); however, Asn297 may also be located about ±3 amino acids upstream or downstream of position 297, i.e., between positions 294 and 300, due to minor sequence variations in Fc region sequences. Such fucosylation variants may have improved ADCC function. In some embodiments, to reduce glycan heterogeneity, the Fc region can be further engineered to replace the Asparagine at position 297 with Alanine (N297A).

In some embodiments, the binding affinity between PD-L1 (e.g., human PD-L1, monkey PD-L1, mouse PD-L1, or extracellular domains thereof) and the engineered PD-1 variants and/or protein constructs as described herein is at least 1-fold, 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, or 50-fold as compared to that between PD-L1 and a wild-type PD-1 or protein constructs thereof.

In some embodiments, the main peak of HPLC-SEC accounts for at least 90%, 95%, 96%, 97%, 98%, 99%, or 99.5% of the engineered PD-1 variants and/or protein constructs as described herein after purification by a protein A column.

In some embodiments, the engineered PD-1 variants and/or protein constructs thereof as described herein can bind to human PD-L1-expressing tumor cells (e.g., human PD-L1 tf CHO—S cells) with an affinity that is at least 1-fold, 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, or 50-fold as compared to that a wild-type PD-1 or protein constructs thereof.

In some embodiments, the engineered PD-1 variants and/or protein constructs thereof as described herein can bind to PD-L1-expressing tumor cells (e.g., PD-L1 tf CHO—S cells) with an affinity that is at least 50%, at least 60%, at least 70%, at least 80%, at least 90%, at least 100%, at least 110%, at least 120%, at least 130%, at least 140%, or at least 150% as compared to the wildetype PD-1.

In some embodiments, the engineered PD-1 variants and/or protein constructs thereof as described herein can block the interaction between human PD-L1 and human PD-1. In some embodiments, the engineered PD-1 variants and/or protein constructs thereof as described herein can block the interaction between human PD-L1-expressing cells (e.g., PD-L1 tf CHO—S cells) and human PD-1. In some embodiments, the blocking ability of the engineered PD-1 variants and/or protein constructs thereof as described herein is at least at least 50%, at least 60%, at least 70%, at least 80%, at least 90%, at least 100%, at least 110%, at least 120%, at least 130%, at least 140%, or at least 150% as compared to that of the wildetype PD-1 or an anti-PD-L1 reference antibody. In some embodiments, the blocking ability of the engineered PD-1 variants and/or protein constructs thereof as described herein is at least 1-fold, 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, or 50-fold as compared to that a wild-type PD-1 or protein constructs thereof.

### Methods of Making Engineered PD-1 Variants and Protein Constructs

Variants of the PD-1 described herein can be prepared by introducing appropriate nucleotide changes into the DNA encoding a PD-1 peptide or a part thereof or by peptide synthesis. Such variants include, for example, deletions, insertions, or substitutions of residues within the amino acids sequences.

Screening can be performed. In a population of such variants, some engineered PD-1 variants will have increased affinity for PD-L1. Any combination of deletions, insertions, and/or combinations can be made to arrive at a variant that has increased binding affinity for the target. The amino acid changes introduced into the variant can also alter or introduce new post-translational modifications into the polypeptide, such as changing (e.g., increasing or decreasing) the number of glycosylation sites, changing the type of glycosylation site (e.g., changing the amino acid sequence such that a different sugar is attached by enzymes present in a cell), or introducing new glycosylation sites.

Engineered PD-1 variants can be derived from any species of animal, including mammals. Non-limiting examples of PD-1 variants include PD-1 variants derived from humans, primates, e.g., monkeys and apes, cows, pigs, horses, sheep, camelids (e.g., camels and llamas), chicken, goats, and rodents (e.g., rats, mice, hamsters and rabbits).

The present disclosure also provides recombinant vectors (e.g., an expression vectors) that include an isolated polynucleotide disclosed herein (e.g., a polynucleotide that encodes a polypeptide disclosed herein), host cells into which are introduced the recombinant vectors (i.e., such that the host cells contain the polynucleotide and/or a vector comprising the polynucleotide), and the production of recombinant polypeptides or fragments thereof by recombinant techniques.

As used herein, a “vector” is any construct capable of delivering one or more polynucleotide(s) of interest to a host cell when the vector is introduced to the host cell. An “expression vector” is capable of delivering and expressing the one or more polynucleotide(s) of interest as an encoded polypeptide in a host cell into which the expression vector has been introduced. Thus, in an expression vector, the polynucleotide of interest is positioned for expression in the vector by being operably linked with regulatory elements such as a promoter, enhancer, and/or a poly-A tail, either within the vector or in the genome of the host cell at or near or flanking the integration site of the polynucleotide of interest such that the polynucleotide of interest will be translated in the host cell introduced with the expression vector.

A vector can be introduced into the host cell by methods known in the art, e.g., electroporation, chemical transfection (e.g., DEAE-dextran), transformation, transfection, and infection and/or transduction (e.g., with recombinant virus). Thus, non-limiting examples of vectors include viral vectors (which can be used to generate recombinant virus), naked DNA or RNA, plasmids, cosmids, phage vectors, and DNA or RNA expression vectors associated with cationic condensing agents.

In some implementations, a polynucleotide disclosed herein (e.g., a polynucleotide that encodes a polypeptide disclosed herein) is introduced using a viral expression system (e.g., vaccinia or other pox virus, retrovirus, or adenovirus), which may involve the use of a non-pathogenic (defective), replication competent virus, or may use a replication defective virus. Techniques for incorporating DNA into such expression systems are well known to those of ordinary skill in the art. The DNA may also be “naked.” The uptake of naked DNA may be increased by coating the DNA onto biodegradable beads that are efficiently transported into the cells.

For expression, the DNA insert comprising a polypeptide-encoding polynucleotide disclosed herein can be operatively linked to an appropriate promoter (e.g., a heterologous promoter), such as the phage lambda PL promoter, the E. coli lac, trp and tac promoters, the SV40 early and late promoters and promoters of retroviral LTRs, to name a few. Other suitable promoters are known to the skilled artisan. In some embodiments, the promoter is a cytomegalovirus (CMV) promoter. The expression constructs can further contain sites for transcription initiation, termination and, in the transcribed region, a ribosome binding site for translation. The coding portion of the mature transcripts expressed by the constructs may include a translation initiating at the beginning and a termination codon (UAA, UGA, or UAG) appropriately positioned at the end of the polypeptide to be translated.

As indicated, the expression vectors can include at least one selectable marker. Such markers include dihydrofolate reductase or neomycin resistance for eukaryotic cell culture and tetracycline or ampicillin resistance genes for culturing in E. coli and other bacteria. Representative examples of appropriate hosts include, but are not limited to, bacterial cells, such as E. coli, Streptomyces, and Salmonella typhimurium cells; fungal cells, such as yeast cells; insect cells such as Drosophila S2 and Spodoptera Sf9 cells; animal cells such as CHO, COS, Bowes melanoma, and HK 293 cells; and plant cells. Appropriate culture mediums and conditions for the host cells described herein are known in the art.

Non-limiting vectors for use in bacteria include pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Non-limiting eukaryotic vectors include pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Non-limiting bacterial promoters suitable for use include the E. coli lacl and lacZ promoters, the T3 and T7 promoters, the gpt promoter, the lambda PR and PL promoters and the trp promoter. Suitable eukaryotic promoters include the CMV immediate early promoter, the HSV thymidine kinase promoter, the early and late SV40 promoters, the promoters of retroviral LTRs, such as those of the Rous sarcoma virus (RSV), and metallothionein promoters, such as the mouse metallothionein-I promoter.

In the yeast Saccharomyces cerevisiae, a number of vectors containing constitutive or inducible promoters such as alpha factor, alcohol oxidase, and PGH can be used.

Introduction of the construct into the host cell can be affected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986), which is incorporated herein by reference in its entirety.

Transcription of DNA encoding a polypeptide of the present disclosure by higher eukaryotes may be increased by inserting an enhancer sequence into the vector. Enhancers are cis-acting elements of DNA, usually about from 10 to 300 bp that act to increase transcriptional activity of a promoter in a given host cell-type. Examples of enhancers include the SV40 enhancer, which is located on the late side of the replication origin at base pairs 100 to 270, the cytomegalovirus early promoter enhancer, the polyoma enhancer on the late side of the replication origin, and adenovirus enhancers.

For secretion of the translated protein into the lumen of the endoplasmic reticulum, into the periplasmic space or into the extracellular environment, appropriate secretion signals may be incorporated into the expressed polypeptide. The signals may be endogenous to the polypeptide or they may be heterologous signals.

The polypeptide (e.g., PD-1 variants) can be expressed in a modified form, such as a fusion protein (e.g., a GST-fusion) or with a histidine-tag, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties can be added to the polypeptide to facilitate purification. Such regions can be removed prior to final preparation of the polypeptide. The addition of peptide moieties to polypeptides to engender secretion or excretion, to improve stability and to facilitate purification, among others, are familiar and routine techniques in the art.

### Methods of Treatment

The engineered PD-1 variants and/or protein constructs of the present disclosure can be used for various therapeutic purposes.

In one aspect, the disclosure provides methods for treating a cancer in a subject, methods of reducing the rate of the increase of volume of a tumor in a subject over time, methods of reducing the risk of developing a metastasis, or methods of reducing the risk of developing an additional metastasis in a subject. In some embodiments, the treatment can halt, slow, retard, or inhibit progression of a cancer. In some embodiments, the treatment can result in the reduction of in the number, severity, and/or duration of one or more symptoms of the cancer in a subject.

In one aspect, the disclosure features methods that include administering a therapeutically effective amount of engineered PD-1 variants and/or protein constructs disclosed herein to a subject in need thereof (e.g., a subject having, or identified or diagnosed as having, a cancer), e.g., breast cancer (e.g., triple-negative breast cancer), carcinoid cancer, cervical cancer, endometrial cancer, glioma, head and neck cancer, liver cancer, lung cancer, small cell lung cancer, lymphoma, melanoma, ovarian cancer, pancreatic cancer, prostate cancer, renal cancer, colorectal cancer, gastric cancer, testicular cancer, thyroid cancer, bladder cancer, kidney cancer, urethral cancer, or hematologic malignancy. In some embodiments, the cancer is melanoma, pancreatic carcinoma, mesothelioma, hematological malignancies, especially hodgkin lymphoma, lymphoma, chronic lymphocytic leukemia, or advanced solid tumors. In some embodiments, the cancer is melanoma, non-small cell lung carcinoma (NSCLC), small cell lung cancer (SCLC), bladder cancer, prostate cancer (e.g., metastatic hormone-refractory prostate cancer), advanced breast cancer, advanced ovarian cancer, and/or advanced refractory solid tumor. In some embodiments, wherein the cancer is melanoma, hodgkin lymphoma, bladder cancer, kidney cancer, breast cancer, lung cancer (e.g., non-small cell lung cancer), head and neck squamous cell cancer, liver cancer, esophageal squamous cell cancer, colorectal cancer, cutaneous squamous cell carcinoma, or merkel cell carcinoma.

In some embodiments, the compositions and methods disclosed herein can be used for treatment of patients at risk for a cancer. Patients with cancer can be identified with various methods known in the art.

As used herein, by an “effective amount” is meant an amount or dosage sufficient to effect beneficial or desired results including halting, slowing, retarding, or inhibiting progression of a disease, e.g., a cancer. An effective amount will vary depending upon, e.g., an age and a body weight of a subject to which the engineered PD-1 variants and/or protein constructs, vector comprising the polynucleotide encoding the engineered PD-1 variants and/or protein constructs, and/or compositions thereof is to be administered, a severity of symptoms and a route of administration, and thus administration can be determined on an individual basis.

An effective amount can be administered in one or more administrations. By way of example, an effective amount of the engineered PD-1 variants and/or protein constructs is an amount sufficient to ameliorate, stop, stabilize, reverse, inhibit, slow and/or delay progression of a cancer in a patient or is an amount sufficient to ameliorate, stop, stabilize, reverse, slow and/or delay proliferation of a cell (e.g., a biopsied cell, any of the cancer cells described herein, or cell line (e.g., a cancer cell line)) in vitro. As is understood in the art, an effective amount may vary, depending on, inter alia, patient history as well as other factors such as the type (and/or dosage) of the engineered PD-1 variants and/or protein constructs used.

Effective amounts and schedules for administering the engineered PD-1 variants and/or protein constructs, the polynucleotides encoding the engineered PD-1 variants and/or protein constructs, and/or compositions disclosed herein may be determined empirically, and making such determinations is within the skill in the art. Those skilled in the art will understand that the dosage that must be administered will vary depending on, for example, the mammal that will receive the engineered PD-1 variants and/or protein constructs, the polynucleotides, and/or compositions disclosed herein, the route of administration, the particular type of polynucleotides, and/or compositions disclosed herein used and other drugs being administered to the mammal.

A typical daily dosage of an effective amount of the engineered PD-1 variants and/or protein constructs is 0.1 mg/kg to 100 mg/kg (mg per kg of patient weight). In some embodiments, the dosage can be less than 100 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, or 0.1 mg/kg. In some embodiments, the dosage can be greater than 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, or 0.1 mg/kg. In some embodiments, the dosage is about 12 mg/kg, 11 mg/kg, 10 mg/kg, 9 mg/kg, 8 mg/kg, 7 mg/kg, 6 mg/kg, 5 mg/kg, 4 mg/kg, 3 mg/kg, 2 mg/kg, or 1 mg/kg. In some embodiments, the dosage is about 1 to 10 mg/kg, about 1 to 5 mg/kg, about 5 to 12 mg/kg, about 3 to 12 mg/kg, or about 2 to 5 mg/kg.

In any of the methods described herein, the engineered PD-1 variants and/or protein constructs can be administered to the subject at least once a week (e.g., once a week, twice a week, three times a week, four times a week, once a day, twice a day, or three times a day).

### Pharmaceutical Compositions and Routes of Administration

Also provided herein are pharmaceutical compositions that contain the engineered PD-1 variants and/or protein constructs described herein. The pharmaceutical compositions can be formulated in any manner known in the art.

Pharmaceutical compositions are formulated to be compatible with their intended route of administration (e.g., intravenous, intraarterial, intramuscular, intradermal, subcutaneous, or intraperitoneal). The compositions can include a sterile diluent (e.g., sterile water or saline), a fixed oil, polyethylene glycol, glycerine, propylene glycol or other synthetic solvents, antibacterial or antifungal agents, such as benzyl alcohol or methyl parabens, chlorobutanol, phenol, ascorbic acid, thimerosal, and the like, antioxidants, such as ascorbic acid or sodium bisulfite, chelating agents, such as ethylenediaminetetraacetic acid, buffers, such as acetates, citrates, or phosphates, and isotonic agents, such as sugars (e.g., dextrose), polyalcohols (e.g., mannitol or sorbitol), or salts (e.g., sodium chloride), or any combination thereof. Liposomal suspensions can also be used as pharmaceutically acceptable carriers. Preparations of the compositions can be formulated and enclosed in ampules, disposable syringes, or multiple dose vials. Where required (as in, for example, injectable formulations), proper fluidity can be maintained by, for example, the use of a coating, such as lecithin, or a surfactant. Absorption of the agents can be prolonged by including an agent that delays absorption (e.g., aluminum monostearate and gelatin). Alternatively, controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid).

Compositions containing the engineered PD-1 variants and/or protein constructs described herein can be formulated for parenteral (e.g., intravenous, intraarterial, intramuscular, intradermal, subcutaneous, or intraperitoneal) administration in dosage unit form (i.e., physically discrete units containing a predetermined quantity of active compound for ease of administration and uniformity of dosage).

Pharmaceutical compositions for parenteral administration are preferably sterile and substantially isotonic and manufactured under Good Manufacturing Practice (GMP) conditions. Pharmaceutical compositions can be provided in unit dosage form (i.e., the dosage for a single administration). Pharmaceutical compositions can be formulated using one or more physiologically acceptable carriers, diluents, excipients or auxiliaries. The formulation depends on the route of administration chosen. For injection, the engineered PD-1 variants and/or protein constructs can be formulated in aqueous solutions, preferably in physiologically-compatible buffers to reduce discomfort at the site of injection. The solution can contain formulatory agents such as suspending, stabilizing and/or dispersing agents. Alternatively the engineered PD-1 variants and/or protein constructs can be in lyophilized form for constitution with a suitable vehicle, e.g., sterile pyrogen-free water, before use.

Toxicity and therapeutic efficacy of compositions can be determined by standard pharmaceutical procedures in cell cultures or experimental animals (e.g., monkeys). One can, for example, determine the LD50 (the dose lethal to 50% of the population) and the ED50 (the dose therapeutically effective in 50% of the population): the therapeutic index being the ratio of LD50:ED50. Agents that exhibit high therapeutic indices are preferred. Where an agent exhibits an undesirable side effect, care should be taken to minimize potential damage (i.e., reduce unwanted side effects). Toxicity and therapeutic efficacy can be determined by other standard pharmaceutical procedures.

Exemplary doses include milligram or microgram amounts of any of the engineered PD-1 variants and/or protein constructs described herein per kilogram of the subject's weight (e.g., about 1 μg/kg to about 500 mg/kg; about 100 μg/kg to about 500 mg/kg; about 100 μg/kg to about 50 mg/kg; about 10 μg/kg to about 5 mg/kg; about 10 μg/kg to about 0.5 mg/kg; about 1 μg/kg to about 50 μg/kg; about 1 mg/kg to about 10 mg/kg; or about 1 mg/kg to about 5 mg/kg). While these doses cover a broad range, one of ordinary skill in the art will understand that therapeutic agents can vary in their potency, and effective amounts can be determined by methods known in the art. Typically, relatively low doses are administered at first, and the attending health care professional or veterinary professional (in the case of therapeutic application) or a researcher (when still working at the development stage) can subsequently and gradually increase the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, and the half-life of the engineered PD-1 variants and/or protein constructs in vivo.

The pharmaceutical compositions can be included in a container, pack, or dispenser together with instructions for administration. The disclosure also provides methods of manufacturing the engineered PD-1 variants and/or protein constructs for various uses as described herein.

### EXAMPLES

The invention is further described in the following examples, which do not limit the scope of the invention described in the claims.

### Example 1. Design of Engineered Human PD-1 Extracellular Domain

The extracellular domain of human PD-1 (hPD-1) belongs to the immunoglobulin superfamily. The 3D structure of the hPD-1 extracellular domain is shown in FIG. 1.

A detailed analysis of PD-L1/hPD-1 complex structure was performed. From the structure, it was determined that A129, Y68 and S73 in hPD-1 (SEQ ID NO: 17) are in proximity of glutamate (Glu) or aspartate (Asp) residues in PD-L1. Thus, when these three amino acids are mutated to histidine residues, they can interact with Glu or Asp residues in PD-L1. From the analysis, it has been determined that additional N-terminal histidine residues will be in close proximity of E58/E60/D61 of PD-L1 (SEQ ID NO: 18).

Based on the finding above, it was determined that mutations at A129, Y68 and S73 can be important to increase the binding affinity of hPD-1 to PD-L1. Further, adding additional N-terminal histidine residues can be important to increase the binding affinity of hPD-1 to PD-L1.

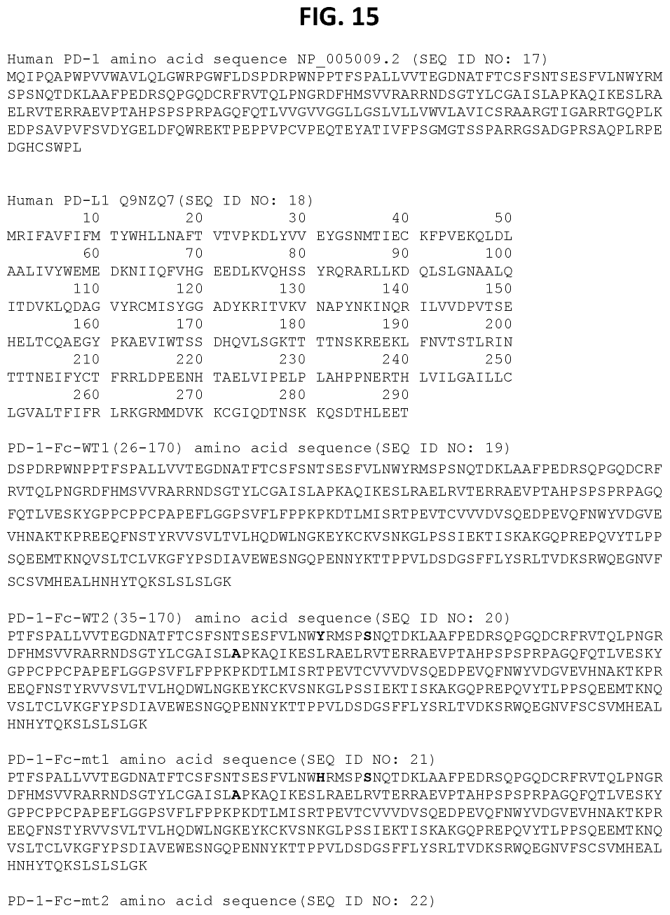

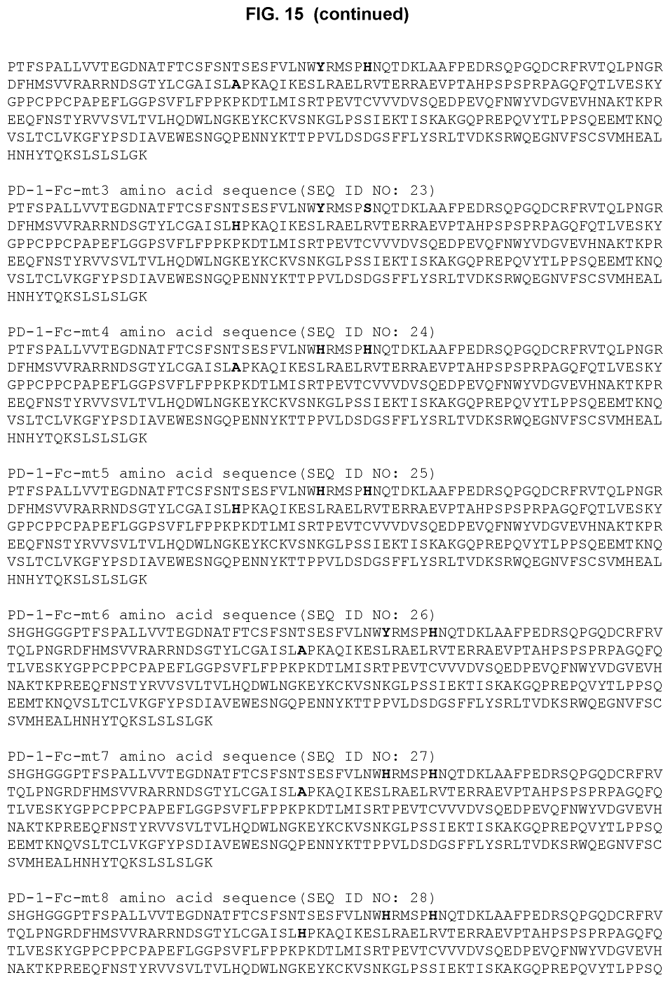

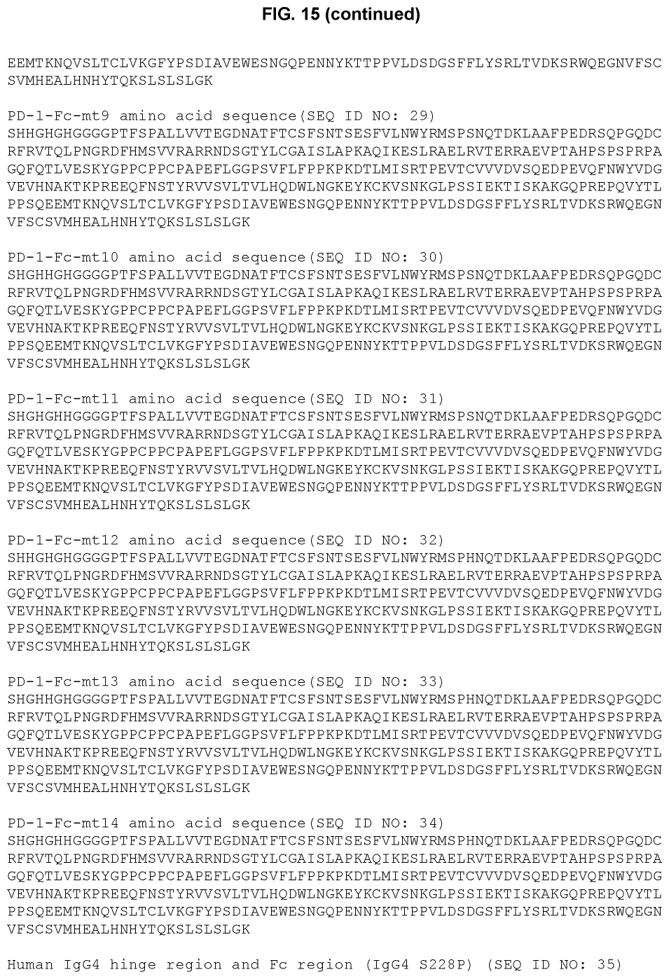

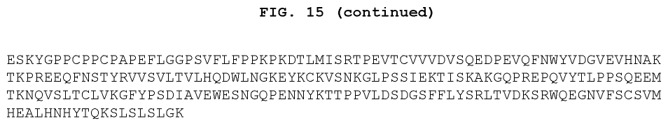

In order to screen hPD-1 extracellular domain mutants having higher blocking activity than wild-type hPD-1 to hPD-L1 (e.g., with different binding affinity to hPD-L1), selected residues were mutated. The amino acid position is based on the sequence of wildtype hPD-1 (SEQ ID NO: 17). PD1-Fc-mts were designed according to the sequences listed in FIG. 2 and FIG. 15.

### Example 2. Production and CMC Analysis for PD1-Fc Mutants (PD1-Fc-Mts)

PD1-Fc-mts were expressed according to the sequences listed in FIG. 15.

The expressed proteins were purified by a protein A column, followed by HPLC-SEC (high-performance liquid chromatography coupled with size exclusion chromatography; Agilent), and the percentage of high molecular weight peaks (HMW %), the percentage of the major peak (Major %), and the percentage of low molecular weight peaks (LMW %) were measured. The amino acid sequences of PD1-Fc-mts were analyzed using the deimmunization tool (Immune Epitope Database And Analysis Resource; Dhanda et al. “Development of a strategy and computational application to select candidate protein analogues with reduced HLA binding and immunogenicity.” Immunology 153.1 (2018): 118-132) to identify immunogenic regions. No immunogenicity was identified.

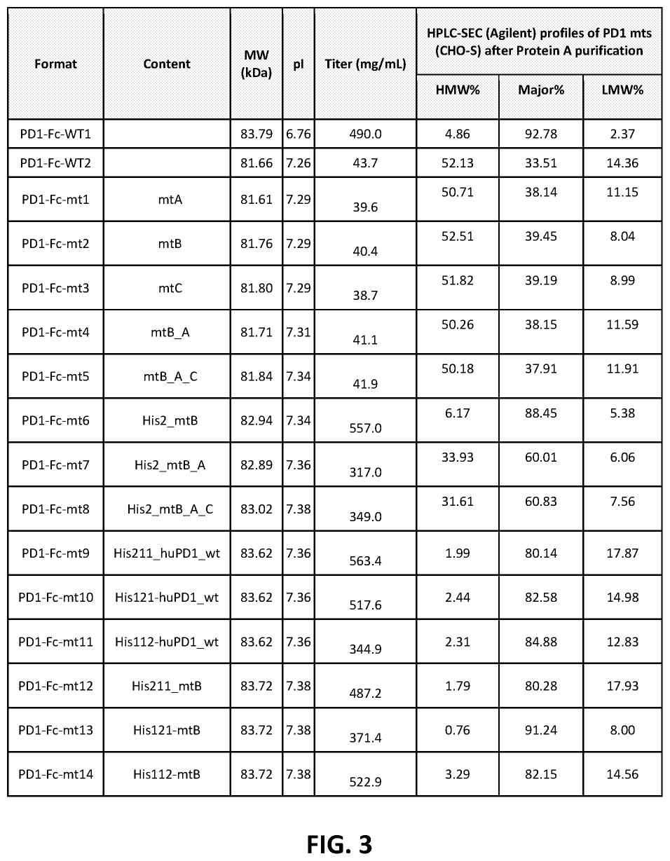

FIG. 3 is a summary of production and CMC analysis results. mtA refers to mutation generated from Y68 to H68; mtB refers to mutation generated from S73 to H73; mtC refers to mutation generated from A129 to H129. His2 indicated the N-terminus of PD1-Fc was extended with amino acid sequence SHGHGGG (SEQ ID NO: 36); His211 indicated the N-terminus of PD1-Fc was extended with amino acid sequence SHHGHGHGGGG (SEQ ID NO: 37); His121 indicated the N-terminus of PD1-Fc was extended with amino acid sequence SHGHHGHGGGG (SEQ ID NO: 38); His112 indicated the N-terminus of PD1-Fc was extended with amino acid sequence SHGHGHHGGGG (SEQ ID NO: 39). HMW % indicated the percentage of high-molecular weight species in the PD1-Fc-mt sample after protein A purification. Major % indicated the percentage of major molecular weight species in the PD1-Fc-mt sample after protein A purification. LMW % indicated the percentage of low-molecular weight species in the PD1-Fc-mt sample after protein A purification.

The production titer was determined by high sensitivity (hs) IgG Assay kit (4BioCell, cat. no.: 200112, 800312) using CuBiAnXC (OPTOCELL technology). The HPLC SEC profiles was determined and analyzed by Waters E2695 Separations Module (GenTECH scientific) with AdvanceBio SEC 2.7 um Columns (Crawfordscientific).

The data in FIG. 3 showed that PD1-Fc-WT2, PD1-Fc-mt1˜mt5 possess lower production titer and lower major molecular weight species than PD1-Fc-WT1 and PD1-Fc-mt6˜mt14. The lower protein quality of PD1-Fc-WT2 and PD1-Fc-mt1˜mt5 will be not used for further in vitro functional analysis.

The data suggested that N-terminal amino acid sequence DSPDRPWNP (SEQ ID NO: 40) or N-terminal extensions with histidine residues enhanced the production titer and major molecular weight species in the PD1-Fc-mt.

### Example 3. Binding Affinity Determination of hPD-1-Fc to PD-L1-ECD-his

PD-L1 binding affinity was determined by Octet Red96 (Forebio). Human PD-L1-ECD-His (Sino cat. no.: I-10084-H08H) was immobilized on anti-Penta-HIS (HIS1K) Biosensor (Fortebio, cat. No. 18-5120). Serial diluted PD1-Fc-mts from 3.7 mg/mL to 300 mg/mL in the assay buffer (1×PBS containing 0.05% Tween-20 with 0.1% BSA at pH7.4 or 1×PBS containing 0.05% Tween-20 with 0.1% BSA at pH6.0) were used as analytes for the biosensor. Binding kinetics were evaluated using the 1:1 Langmuir binding model in Fortebio Data Analysis 11.0 Software.

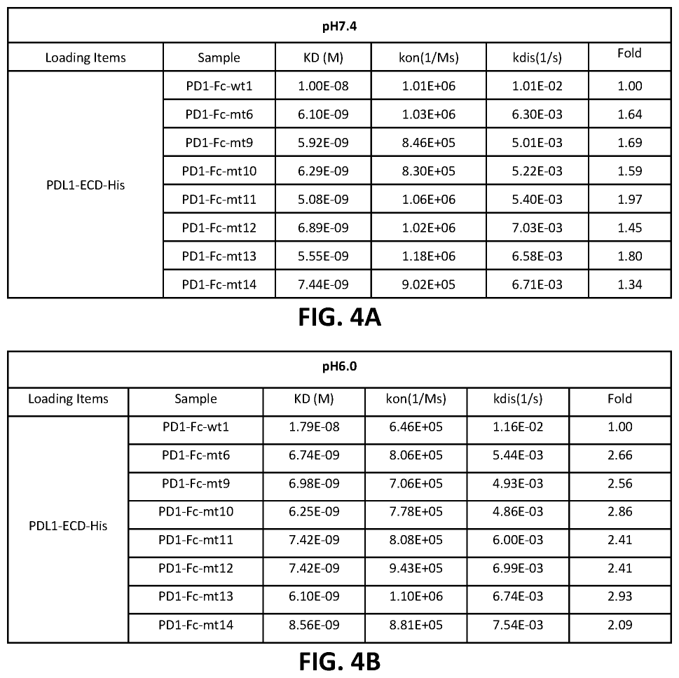

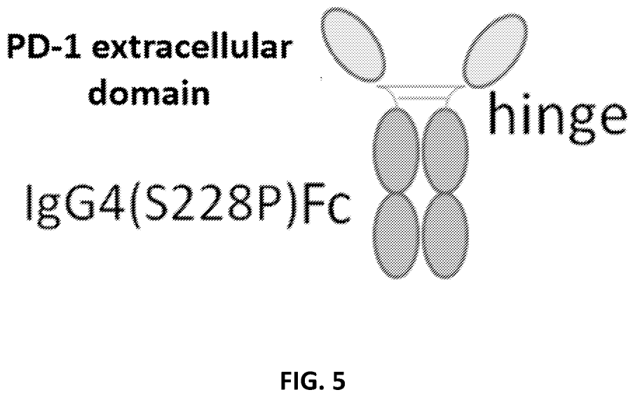

As shown in FIGS. 4A-4B, all the tested PD1-Fc-mts had better binding affinity to PD-L1-ECD-His than PD1-Fc-WT1 at both pH7.4 and pH6.0. PD1-Fc-mt11 possess the best binding affinity to PD-L1-ECD-His at pH7.4 among the tested molecules. PD1-Fc-mt13 possess the best binding affinity to PD-L1-ECD-His at pH6.0 among the tested molecules.

### Example 4. Whole Cell Bind Assays to PD-L1 Transfected CHO—S and PD-L1-Expressing Tumor Cells

Whole cell binding ability of PD1-Fc-mts were tested by incubating the PD-L1 transfected CHO—S cells with the serially diluted PD1-Fc-mts in the modified FACS buffer (0.1M Phosphate buffer containing 4% FBS at pH7.3-7.4 or pH5.8-6.0) at 4° C. for 30 minutes. The cells were washed with FACS buffer (1×PBS containing 4% FBS) and the binding was detected with goat anti-human Fc PE Ab (Invitrogen, cat. no: 109-115-098) at 4° C. for another 30 minutes. Flow cytometry analyses were performed using the CytoFLEX (Beckman Coulter Inc.).

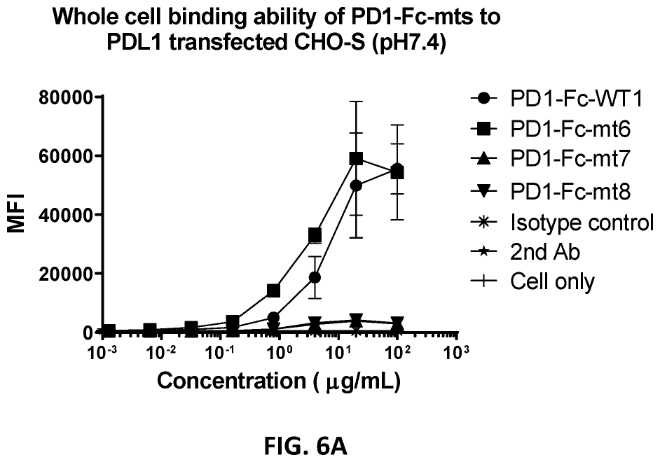

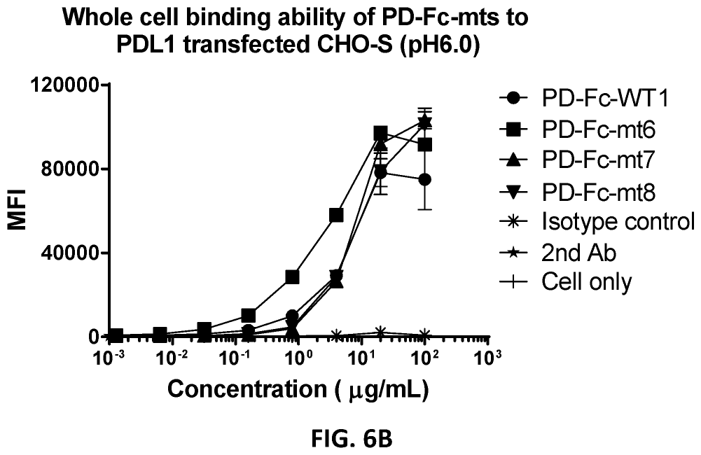

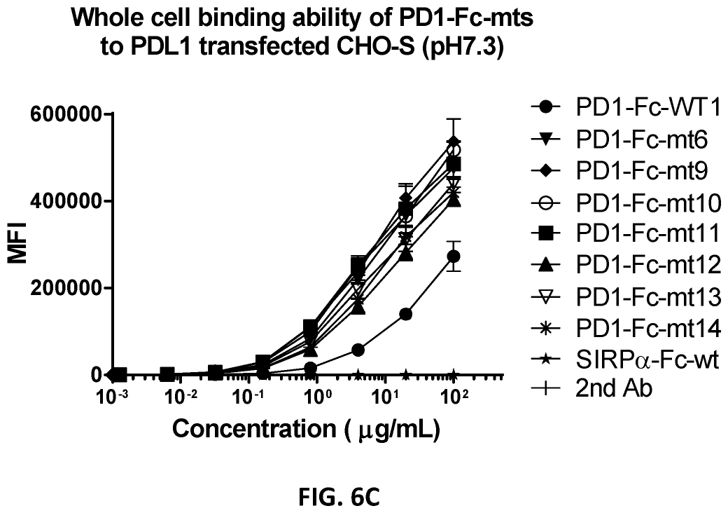

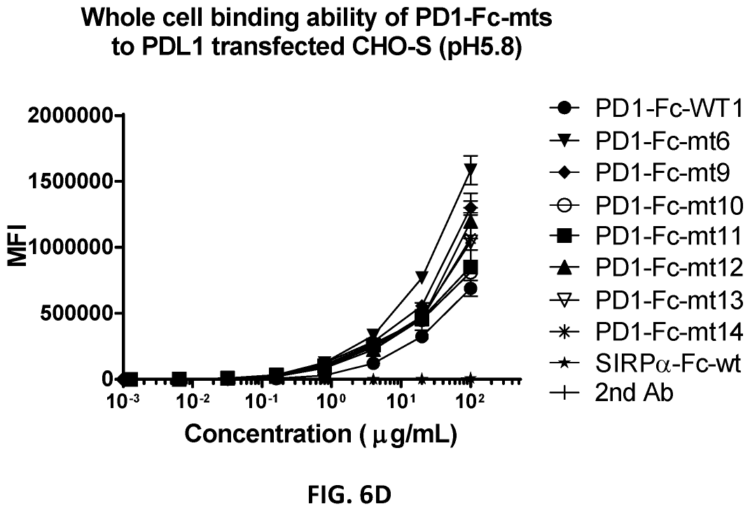

PD1-Fc-WT1 was used as the positive control. Isotype control (anti-PD-1 Ab) and SIRPa-Fc-wt were used as negative controls. FIGS. 6A and 6C show the PD1-Fc-mts binding to PD-L1 transfected cells at pH7.3-7.4 by flow cytometry; FIGS. 6B and 6D show the PD1-Fc-mts binding to PD-L1 transfected cells at pH5.8-6.0 by flow cytometry. The data indicate that all the selected PD1-Fc-mts can bind to PD-L1 transfected cells. Further, selected PD1-Fc-mts show better binding ability to PDL1 transfected cells at pH5.8-6.0 than at pH7.3-7.4. The data further demonstrate that PD1-Fc-mt6, PD1-Fc-mt9, PD1-Fc-m10, PD1-Fc-mt11, PD1-Fc-mt12, PD1-Fc-mt13, and PD1-Fc-mt14 possess better binding ability to PDL1 transfected CHO—S than PD1-Fc-WT1 at pH7.3-7.4 or pH5.8-6.0.

### Example 5. PD-L2 Binding Assays

ELISA assays were performed to test the binding ability of PD1-Fc-mts to recombinant human PD-L2-ECD-His fusion proteins. 2 μg/ml of anti-His antibody (R&D, cat. no. MAB050-500) were coated on 96-well EIA microplate overnight at 4° C. After blocking with 5% skim milk, 1 μg/ml of recombinant human PDL2 His tag fusion proteins (R&D, cat. no: 9075-PL) were added and incubated at 24° C. for 1 hour. After washing with modified 0.1 M phosphate buffer with pH7.3 or pH5.8 for three times, serially diluted PD1-Fc-mts in modified 0.1 M phosphate buffer with pH7.3 or pH5.8 were added and incubated at 24° C. for 1 hour. The unbound PD1-Fc-mts were removed, and wells were washed with 1×PBST containing 0.1% Tween 20 for three times. The HRP-conjugated secondary antibody (Jackson Immu., cat. No.: 109-035-008) was added to the wells at 24° C. for 1 hour. After incubation, excess secondary antibodies were washed with 1×PBST containing 0.1% Tween 20 for three times. TMB was added to the wells, and following incubation, the reaction was stopped, and HRP activity was measured by spectrophotometer (Varioskan LUK, Thermo Scientific, type3020) at 450 nm.

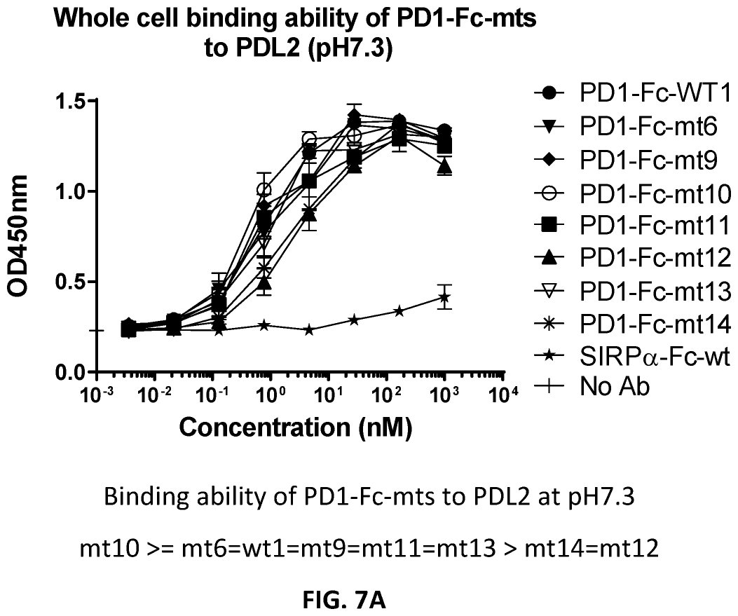

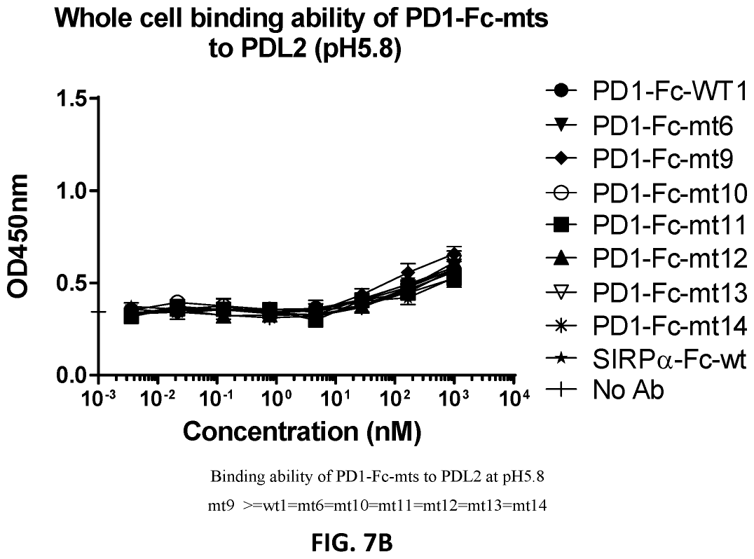

PD1-Fc-WT1 was used as the positive control. SIRPα-Fc-wt was used as the negative control. FIGS. 7A and 7B show the binding affinity between PD1-Fc-mts and recombinant human PD-L2-ECD-His fusion protein at pH7.3 or pH5.8, respectively, as measured by ELISA. The data indicate that all the selected PD1-Fc-mts can bind to PD-L2 at pH7.3 but not at pH5.8. In addition, FIG. 7A shows the PD-L2 binding ability of PD1-Fc-mt6, PD1-Fc-mt9, PD1-Fc-mt10, PD1-Fc-mt11, and PD1-Fc-mt13 were similar as that of PD1-Fc-WT1 at pH7.3. But PD1-Fc-mt12 and PD1-Fc-mt14 exhibit lower binding affinity to PD-L2.

### Example 6. Cross-Species Binding Ability of PD-Fc-Mts to Monkey PD-L1 and Mouse PD-L1

ELISA assays were performed to test the binding ability of PD1-Fc-mts to recombinant Cynomolgus Monkey PD-L1 Fc (R&D, cat.: 9326-B7-100) or mouse PD-L1 His Tag fusion proteins (R&D, cat. No.: 9048-B7-100). One microgram per ml of Cynomolgus Monkey PDL1 or mouse PDL1 were coated on 96-well EIA microplate at 4° C. overnight. After blocking with 5% skim milk, serially diluted biotin-labeled PD1-Fc-mts were added and incubated at 24° C. for 1 hour. The unbound biotin-labeled PD1-Fc-mts were removed, and wells were washed with 1×PBST containing 0.1% Tween 20 for three times. The HRP-conjugated Avidin (Biolegend, cat. No.: 405103) was added to the wells at 24° C. for 1 hour. After incubation, excess secondary antibodies were washed with 1×PBST containing 0.1% Tween 20 for three times. TMB was added to the wells, and following incubation, the reaction was stopped, and HRP activity was measured by spectrophotometer (Varioskan LUK, Thermo Scientific, type3020) at 450 nm.

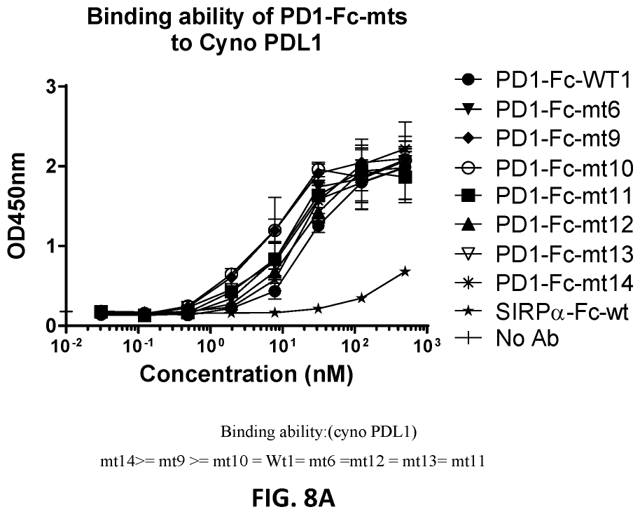

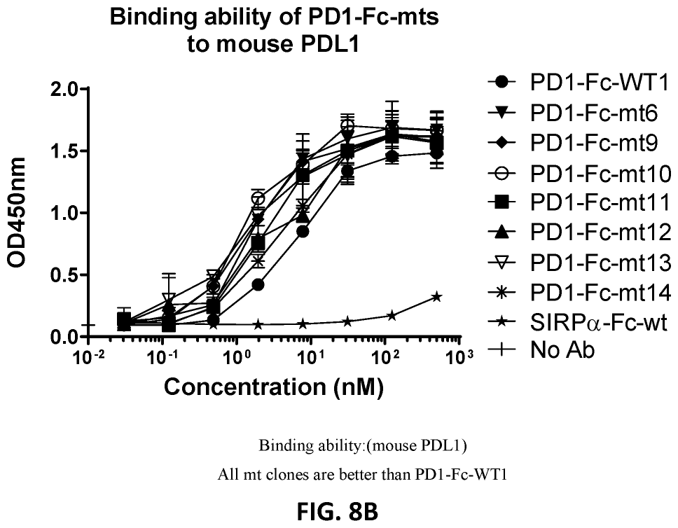

PD1-Fc-WT1 was used as the positive control. SIRPα-Fc-wt was used as the negative control. FIGS. 8A and 8B demonstrated the tested PD1-Fc-mts can bind to recombinant Cynomolgus Monkey PD-L1 Fc fusion proteins and/or recombinant mouse PD-L1 His tag fusion proteins by ELISA, respectively.

### Example 7. Whole Cell Blocking Ability of PD1-Fc-Mts

PDL1 transfected CHO—S cells (2E4 cells/well) were washed and resuspended in modified FACS buffer (0.1 M Phosphate buffer containing at pH7.3˜7.4 or pH5.8˜6.0). Various concentrations of tested PD1-Fc-mts and the fixed concentration of biotin-PD1-Fc-WT1 (1 μg/mL) in modified FACS buffer (0.1 M Phosphate buffer at pH7.3˜7.4 or pH5.8˜6.0) were mixed and co-incubated with cells at 4° C. for 30 minutes. Unbound tested PD1-Fc-mts and biotin-PD1-Fc-WT1 were washed off and then the cells were stained with Strepavidin-PE (eBioscience, cat. no.: EBS12-4317-87) in FACS buffer (1×PBS containing 4% FBS) at 4° C. for 30 minutes. Flow cytometry analyses were performed using the CytoFLEX (Beckman Coulter Inc.). Anti-PDL1 ref. Ab. and isotype control (SIRPα-Fc-wt) were used as the positive and negative control, respectively.

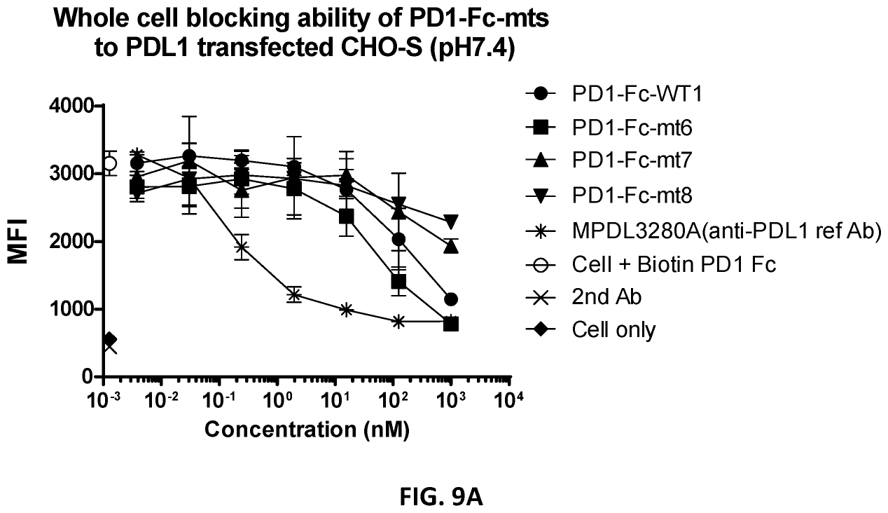

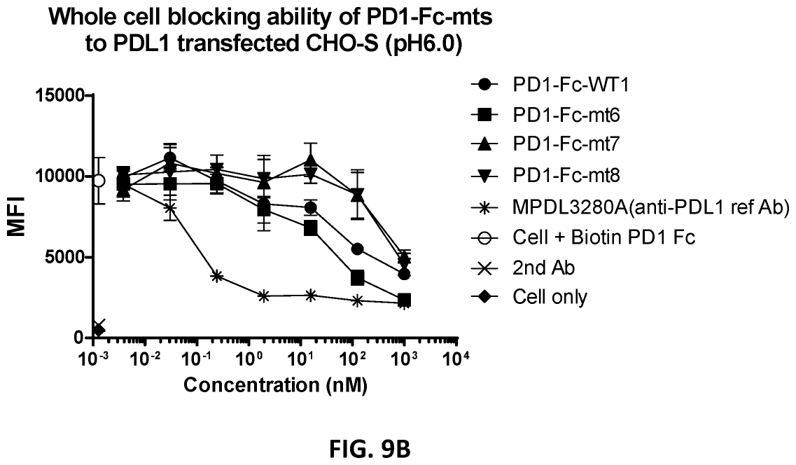

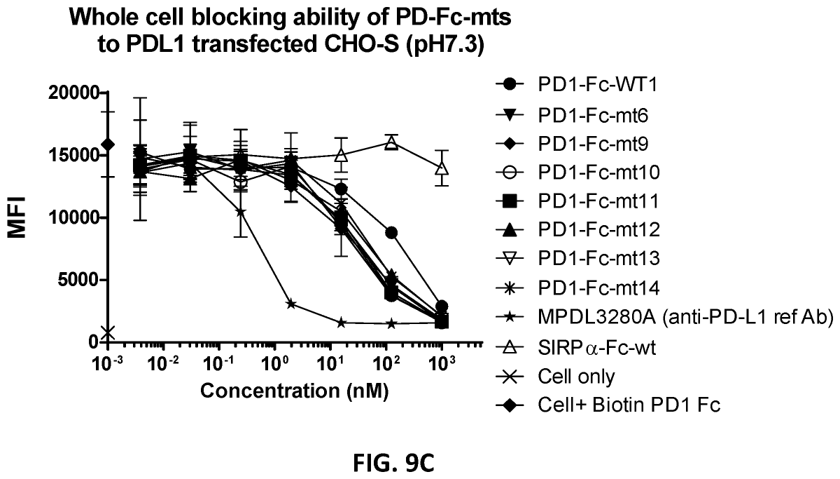

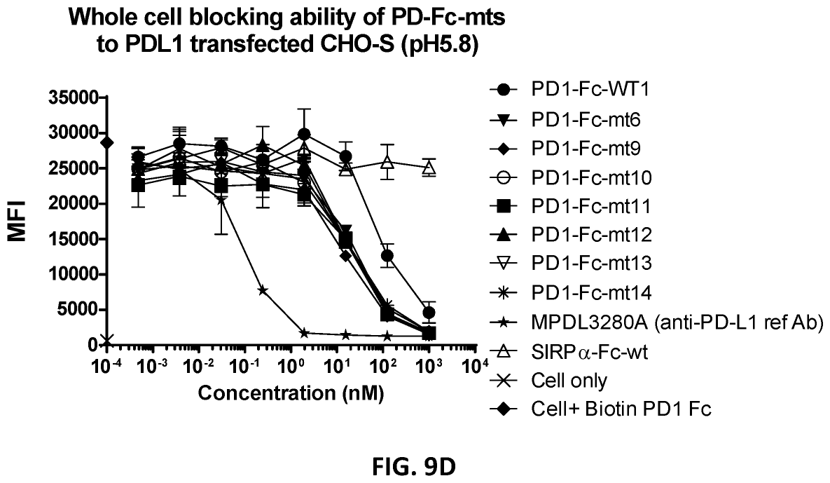

The data presented in FIGS. 9A-9D demonstrated that all the selected PD1-Fc-mts can block PD1-Fc-WT1 binding to PD-L1 transfected CHO—S cells.

FIGS. 9A and 9C show blocking of PD1-Fc-WT1 binding to PDL1 transfected CHO—S at pH7.3˜7.4 by PD1-Fc-mts. FIGS. 9B and 9D show blocking of PD1-Fc-WT1 binding to PDL1 transfected CHO—S at pH5.8˜6.0 by PD1-Fc-mts.

The data further demonstrated that PD1-Fc-mt6, PD1-Fc-mt9, PD1-Fc-m10, PD1-Fc-mt11, PD1-Fc-mt12, PD1-Fc-mt13, and PD1-Fc-mt14 possess better blocking ability than PD1-Fc-WT1 at both pH7.3˜7.4 and pH5.8˜6.0.

### Example 8. Enhancement of T Cell Response in the MLR Assay for PD1-Fc-Mts

Mixed lymphocyte reaction (MLR) assays were performed to determine the enhancement of T cell response by the candidate molecules. T cells were labelled with 5 nM Celltrace violet (Thermo, cat. no. C34557) at 37° C. for 10 minutes, washed by MES-buffered complete RPMI-1640 medium at pH6.5 or pH7.2, twice. 1×10⁵ CellTrace violet labelled CD4+ T cells and 1×10⁴ dendritic cells (DCs) were co-incubated with the candidate molecules at 5 nM, 50 nM, or 500 nM in MES-buffered complete RPMI-1640 medium at pH6.5 or pH7.2. Negative control molecule (SIRPα-Fc-G4) was also used for co-incubation. After 5-days co-incubation, cells were harvested and cell proliferation was analyzed by CytoFLEX-S (Beckman Coulter Inc.). Culture supernatant were also harvested, and IL-2 secretion and IFN-γ secretion were determined by Human IL-2 ELISA MAX Deluxe (Biolegend, cat. no.: 431805) and Human IFNγ ELISA MAX Deluxe (Biolegend, cat. no.: 430105), respectively.

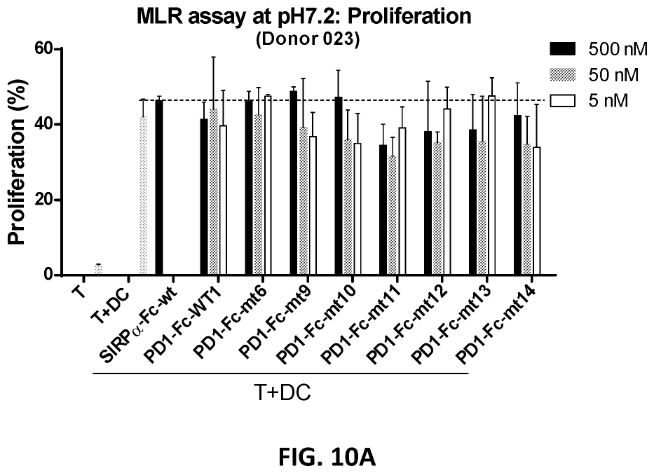

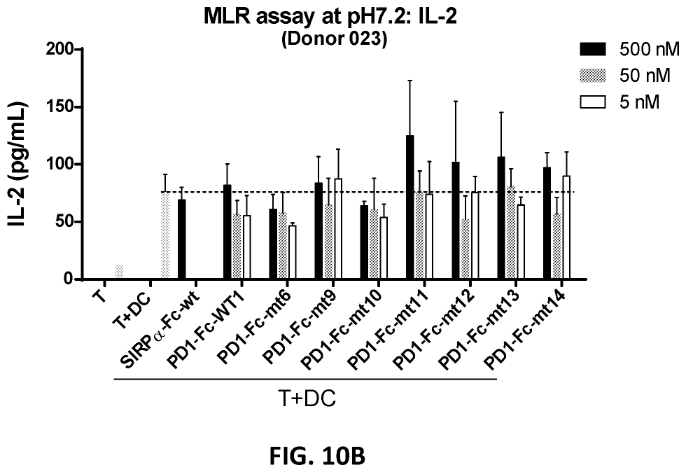

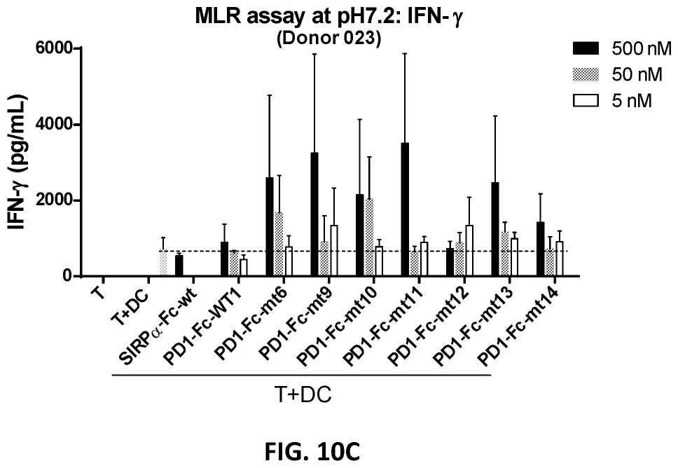

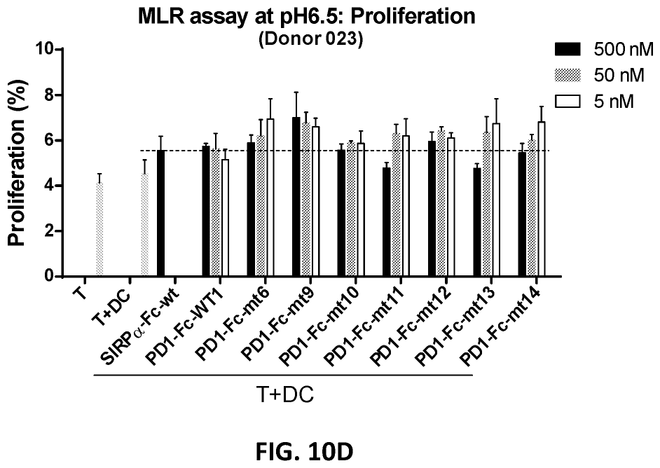

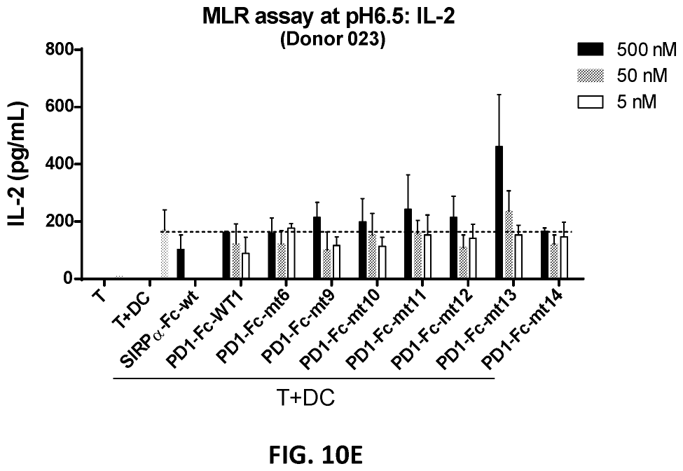

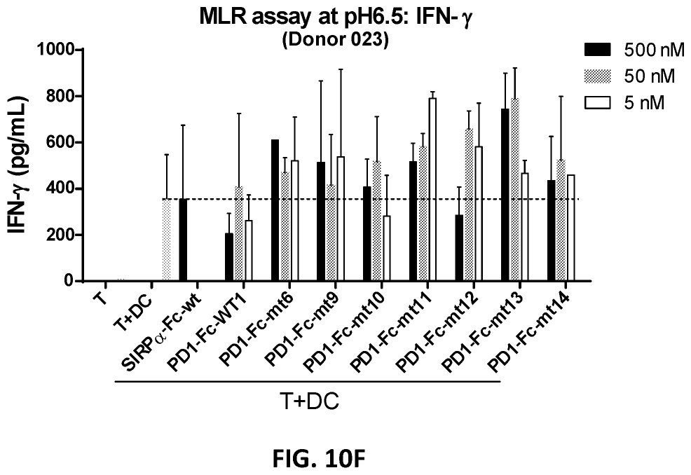

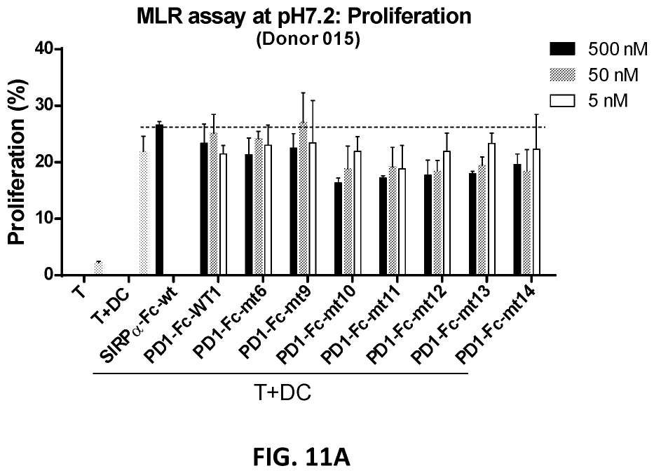

FIGS. 10A-10F show the MLR data from Donor 023.

As shown in FIGS. 10A and 10D, cell proliferation in the MLR assays was determined using the CellTrace™ violet cell proliferation kit (Thermo, cat. no. C34557). Specifically, the percentage of weaker CellTrace™violet-labeled cells (indicating proliferation cells) over the CD3+/7-ADD-cells (indicating total T cells) was calculated. The results show that cells treated with PD1-Fc-mts did not significantly change the cell proliferation as compared to that of the PD1-Fc-WT1.

As shown in FIGS. 10B and 10E, IL-2 secretion in the MLR assays was determined using Human IL-2 ELISA MAX Deluxe (Biolegend, cat. no.: 431805). The results show that PD1-Fc-mt11, PD1-Fc-mt12, PD1-Fc-mt13, and PD1-Fc-mt14 exhibited better IL-2 secretion level than PD1-Fc-WT1 at pH 7.2, and PD1-Fc-mt13 showed the highest IL-2 secretion at pH6.5 among the tested molecules.

As shown in FIGS. 10C and 10F, IFN-γ secretion in the MLR assays was determined using Human IFNγ ELISA MAX Deluxe (Biolegend, cat. no.: 430105). The results show that PD1-Fc-mt6, PD1-Fc-mt9, PD1-Fc-mt10, PD1-Fc-mt11, PD1-Fc-mt13 and PD1-Fc-mt14 exhibited better IFN-γ secretion level than PD1-Fc-WT1 at pH 7.2. All the tested PD1-Fc-mts exhibited better IFN-γ secretion level than PD1-Fc-WT1.

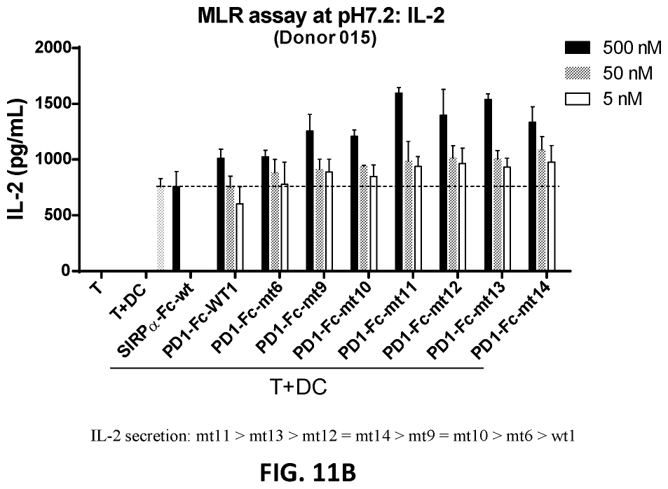

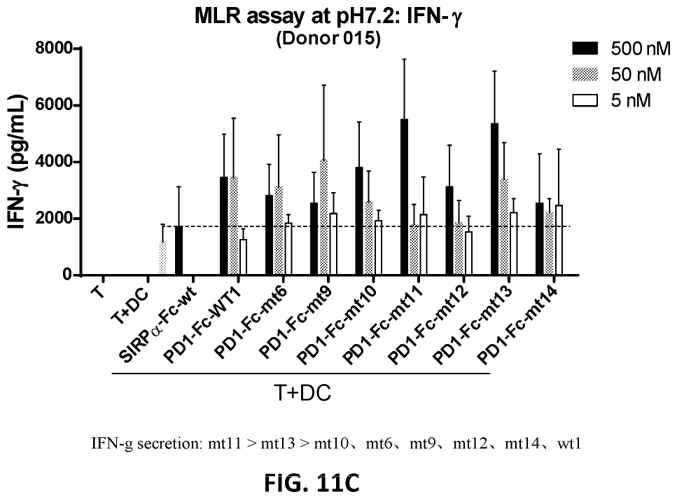

FIGS. 11A-11C show the MLR data from Donor 015.

As shown in FIG. 11A, cell proliferation in the MLR assays was determined using the CellTrace™ violet cell proliferation kit (Thermo, cat. no. C34557). The results show that cells treated with PD1-Fc-mts did not significantly change the cell proliferation as compared to that of the PD1-Fc-WT1.

As shown in FIG. 11B, IL-2 secretion in the MLR assays was determined using Human IL-2 ELISA MAX Deluxe (Biolegend, cat. no.: 431805). The results show that PD1-Fc-mt11, PD1-Fc-mt12, PD1-Fc-mt13, and PD1-Fc-mt14 exhibited better IL-2 secretion level than PD1-Fc-WT1 at pH 7.2.

As shown in FIG. 11C, IFN-γ secretion in the MLR assays was determined using Human IFNγ ELISA MAX Deluxe (Biolegend, cat. no.: 430105). The results show that PD1-Fc-mt11 and PD1-Fc-mt13 exhibited better IFN-γ secretion level than PD1-Fc-WT1 at pH 7.2.

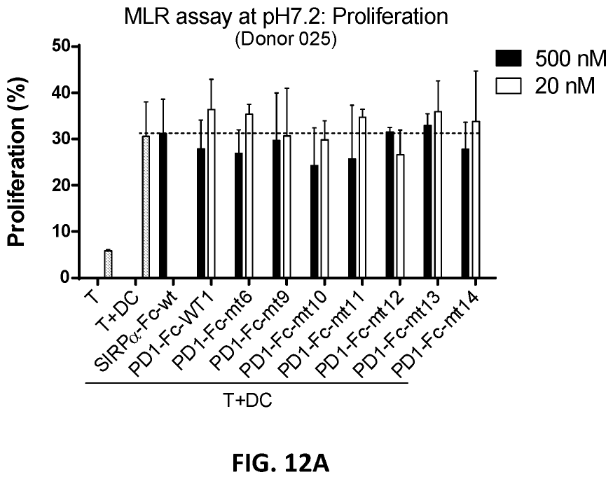

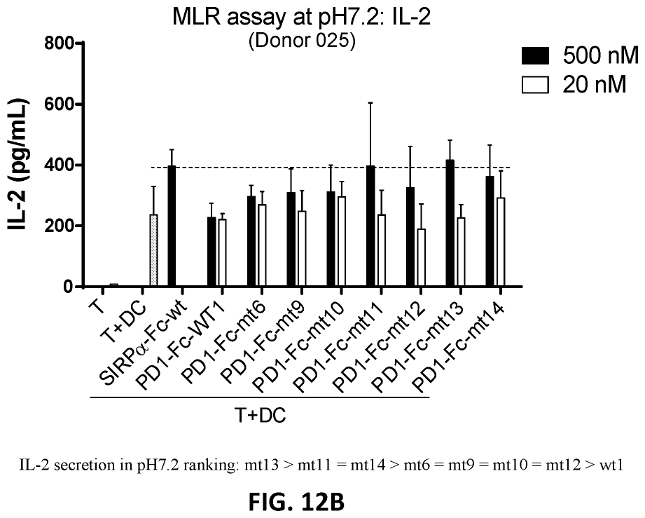

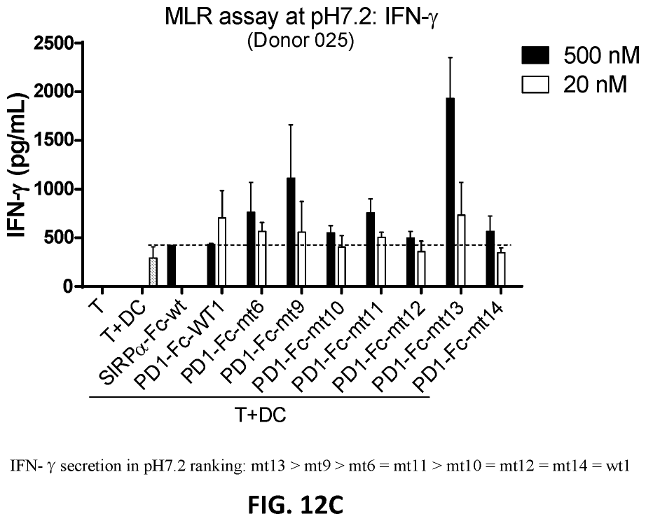

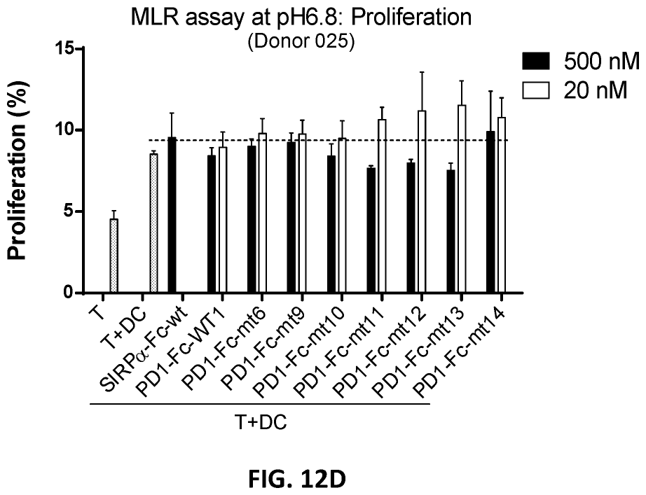

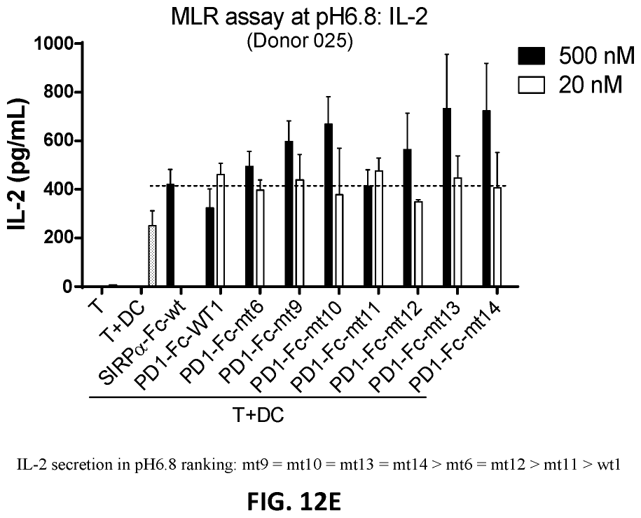

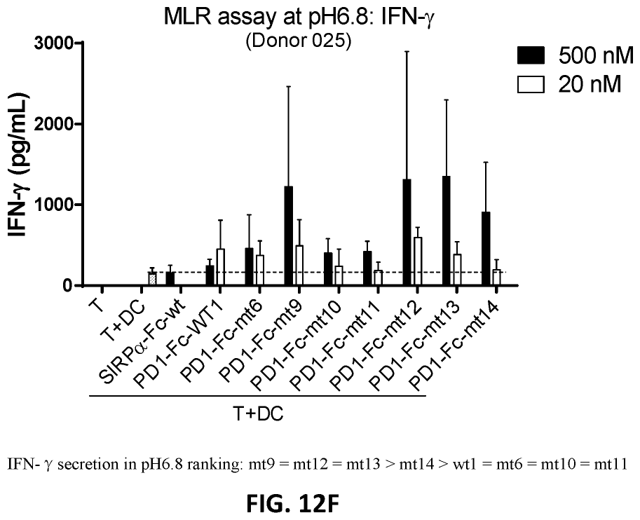

FIGS. 12A-12F show the MLR data from Donor 025.

As shown in FIGS. 12A and 12D, cell proliferation in the MLR assays was determined using the CellTrace™ violet cell proliferation kit (Thermo, cat. no. C34557). The results show that cells treated with PD1-Fc-mts did not significantly change the cell proliferation as compared to that of the PD1-Fc-WT1.

As shown in FIGS. 12B and 12E, IL-2 secretion in the MLR assays was determined using Human IL-2 ELISA MAX Deluxe (Biolegend, cat. no.: 431805). The results show that all the tested PD1-Fc-mts exhibited better IL-2 secretion level than PD1-Fc-WT1 at pH 7.2 and at pH 6.8.

As shown in FIGS. 12C and 12F, IFN-γ secretion in the MLR assays was determined using Human IFNγ ELISA MAX Deluxe (Biolegend, cat. no.: 430105). The results show that PD1-Fc-mt6, PD1-Fc-mt9, PD1-Fc-mt11 and PD1-Fc-mt13 exhibited better IFN-γ secretion level than PD1-Fc-WT1 at pH 7.2. The results show that PD1-Fc-mt9, PD1-Fc-mt12, PD1-Fc-mt13 and PD1-Fc-mt14 exhibited better IFN-γ secretion level than PD1-Fc-WT1 at pH 6.8.

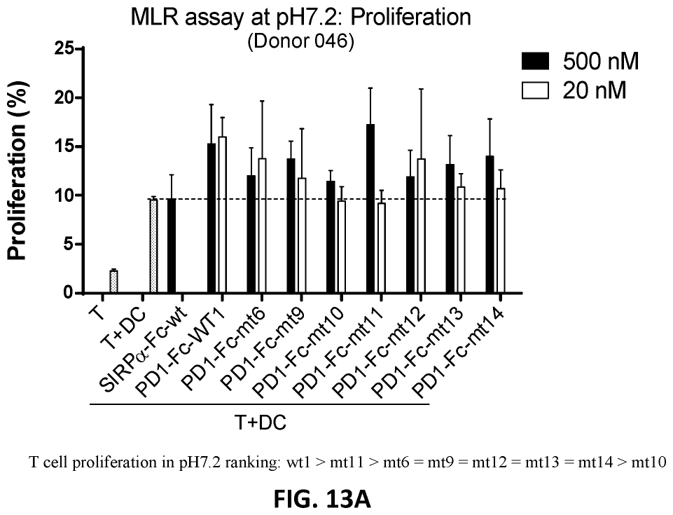

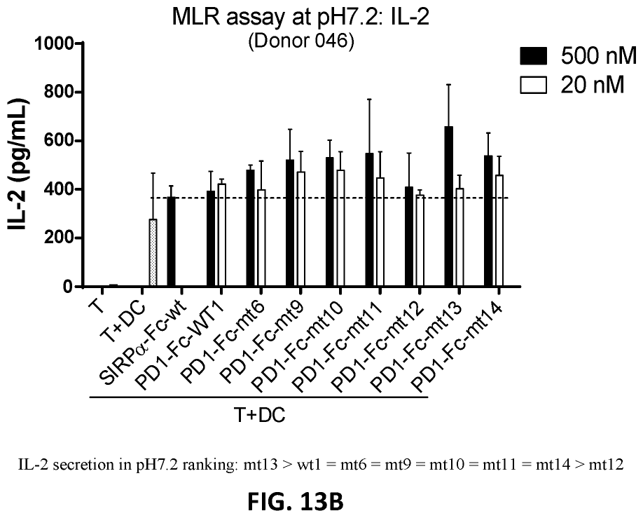

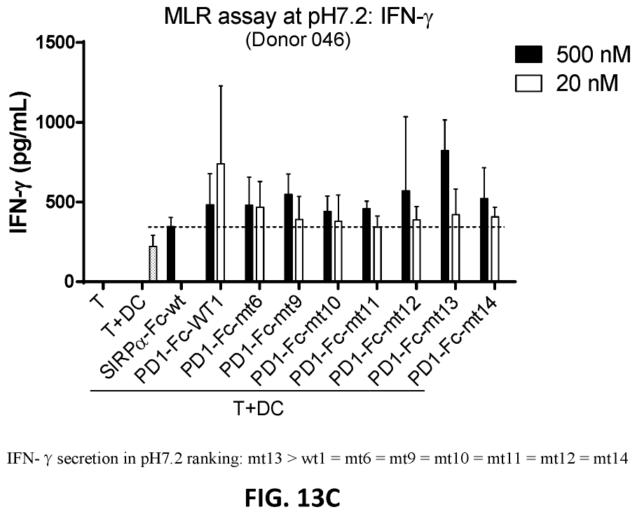

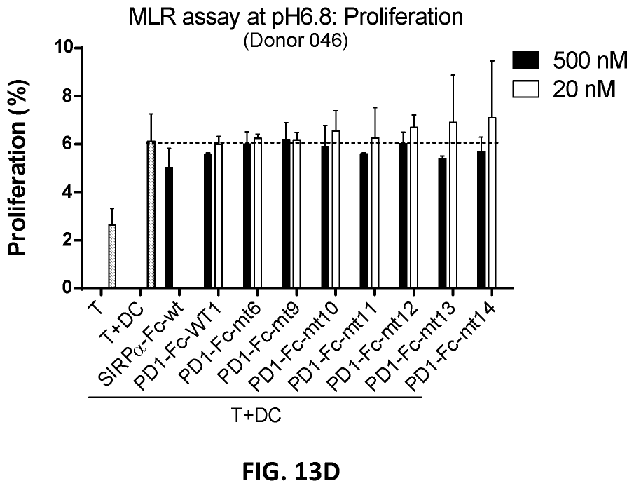

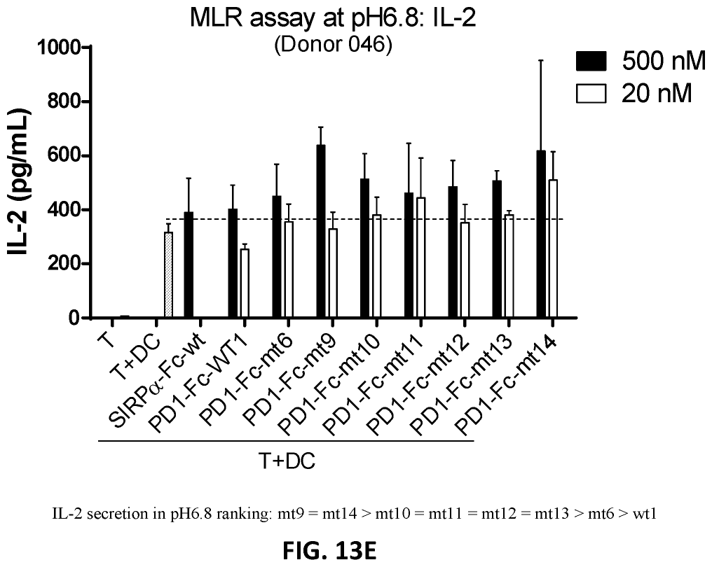

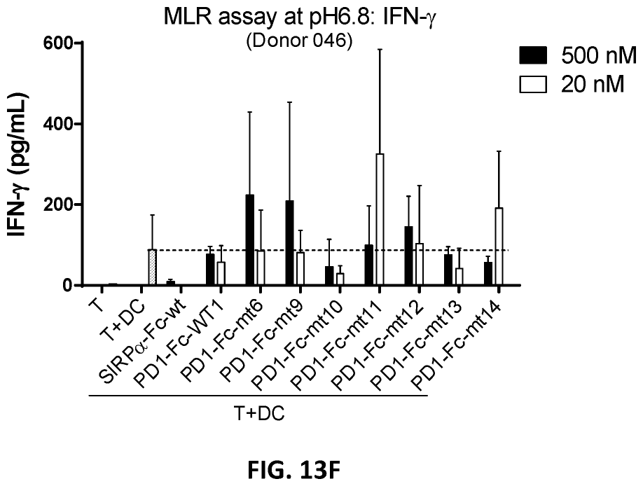

FIGS. 13A-13F show the MLR data from Donor 046.

As shown in FIGS. 13A and 13D, cell proliferation in the MLR assays was determined using the CellTrace™ violet cell proliferation kit (Thermo, cat. no. C34557). The results show that cells treated with PD1-Fc-mts did not significantly change the cell proliferation as compared to that of the PD1-Fc-WT1.

As shown in FIGS. 13B and 13E, IL-2 secretion in the MLR assays was determined using Human IL-2 ELISA MAX Deluxe (Biolegend, cat. no.: 431805). The results show that PD1-Fc-mt13 showed the highest IL-2 secretion at pH 7.2 among the tested molecules, and that all the tested PD1-Fc-mts exhibited better IL-2 secretion level than PD1-Fc-WT1 at pH 6.8.

As shown in FIGS. 13C and 13F, IFN-γ secretion in the MLR assays was determined using Human IFNγ ELISA MAX Deluxe (Biolegend, cat. no.: 430105). The results show that PD1-Fc-mt13 showed the highest IFN-γ secretion at pH 7.2 among the tested molecules. The results show that PD1-Fc-mt6, PD1-Fc-mt9, PD1-Fc-mt11, PD1-Fc-mt12 and PD1-Fc-mt14 exhibited better IFN-γ secretion level than PD1-Fc-WT1 at pH 6.8.

### Example 9. Enhancement of T Cell Response in the MLR Assay for PD1-Fc-Mts

Mixed lymphocyte reaction (MLR) assays were performed at pH7.2 or pH6.5˜6.8 to determine the enhancement of T cell response by the PD1-Fc-mts using T cell isolated from different donors. Cell proliferation in the MLR assays was determined by CellTrace™ violet cell proliferation kit (Thermo, cat. no. C34557). IL-2 and IFN-r secretion level were determined by Human IL-2 ELISA MAX Deluxe (Biolegend, cat. no.: 431805) and Human IFNγ ELISA MAX Deluxe (Biolegend, cat. no.: 430105), respectively.

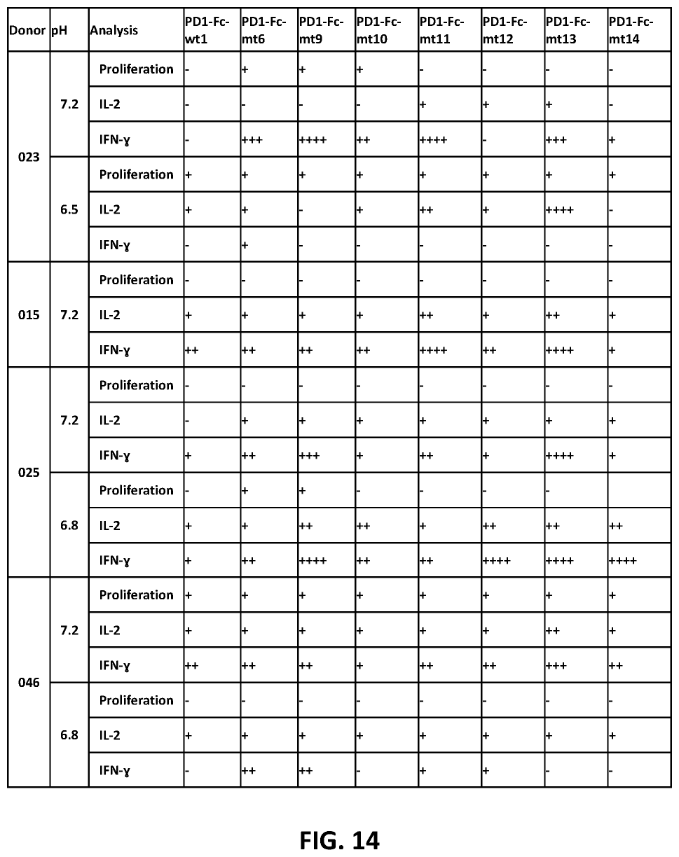

The data were summarized in FIG. 14. Compared to the T+DC group, the relative fold enhancement of PD1-Fc-mts at 500 nM was graded. “−” indicated the enhancement fold relative to T+DC group is 0 to 1 fold; “+” indicated the enhancement fold relative to T+DC group is 1 to 2 folds; “++” indicated the enhancement fold relative to T+DC group is 2 to 3 folds. “+++” indicated the enhancement fold relative to T+DC group is 3 to 4 folds; “++++” indicated the enhancement fold relative to T+DC group is greater than 4 folds.

According to the summarized table, PD1-Fc-mt6, PD1-Fc-mt9, PD1-Fc-mt11, and PD1-Fc-mt13 exhibited the better T cell response in the MLR assay. Especially, PD1-Fc-mt13 show the best T cell responses in the MLR assay among the tested molecules.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An engineered polypeptide comprising a PD-1 extracellular region and a PD-L1 surface interaction sequence with at least 5 amino acids, wherein the PD-L1 surface interaction sequence comprises two or more positively charged amino acids.

2. The engineered polypeptide of claim 1, wherein the positively charged amino acids are selected from the group consisting of: histidine, arginine and lysine.

3. The engineered polypeptide of claim 1 or 2, wherein the PD-L1 surface interaction sequence has an overall positive charge.

4. The engineered polypeptide of any one of claims 1-3, wherein the off rate between the engineered polypeptide and PD-L1 is lower than the off rate between the PD-1 extracellular region without the PD-L1 surface interaction sequence and PD-L1.

5. The engineered polypeptide of any one of claims 1-4, wherein the PD-1 extracellular region comprises an amino acid sequence that is at least 80% identical to SEQ ID NO: 16.

6. The engineered polypeptide of any one of claims 1-5, wherein the PD-L1 surface interaction sequence is at the N-terminus of the engineered polypeptide.

7. The engineered polypeptide of any one of claims 1-6, wherein the PD-L1 surface interaction sequence comprise a sequence that is at least 80% identical to SHGHGGG, SHHGHGHGGGG, SHGHHGHGGGG or SHGHGHHGGGG.

8. The engineered polypeptide of any one of claims 1-7, wherein one or more of the amino acids that corresponds to Y34, S39 and A95 of SEQ ID NO: 16 is H.

9. The engineered polypeptide of any one of claims 1-8, wherein the amino acid that corresponds to Y34 of SEQ ID NO: 16 is H.

10. The engineered polypeptide of any one of claims 1-9, wherein the amino acid that corresponds to S39 of SEQ ID NO: 16 is H.

11. The engineered polypeptide of any one of claims 1-10, wherein the amino acid that corresponds to A95 of SEQ ID NO: 16 is H.

12. The engineered polypeptide of any one of claims 1-11, comprising one of the following:
(a) the amino acid that corresponds to Y34 of SEQ ID NO: 16 is H;
(b) the amino acid that corresponds to S39 of SEQ ID NO: 16 is H;
(c) the amino acid that corresponds to A95 of SEQ ID NO: 16 is H;
(d) the amino acids that correspond to Y34 and S39 of SEQ ID NO: 16 are H;
(e) the amino acids that correspond to Y34 and A95 of SEQ ID NO: 16 are H;
(f) the amino acids that correspond to S39 and A95 of SEQ ID NO: 16 are H;
(g) the amino acids that correspond to Y34, S39 and A95 of SEQ ID NO: 16 are H.

13. The engineered polypeptide of any one of claims 1-12, wherein the PD-L1 surface interaction sequence has 5-15 amino acids.

14. An engineered polypeptide comprising an amino acid sequence that is at least 80% identical to SEQ ID NO: 16, wherein one or more of the amino acids that corresponds to Y34, S39 and A95 of SEQ ID NO: 16 is H.

15. The engineered polypeptide of claim 14, wherein the amino acid that corresponds to Y34 of SEQ ID NO: 16 is H.

16. The engineered polypeptide of any one of claim 14 or 15, wherein the amino acid that corresponds to S39 of SEQ ID NO: 16 is H.

17. The engineered polypeptide of any one of claims 14-16, wherein the amino acid that corresponds to A95 of SEQ ID NO: 16 is H.

18. The engineered polypeptide of any one of claims 14-17, comprising one of the following:
(a) the amino acid that corresponds to Y34 of SEQ ID NO: 16 is H;
(b) the amino acid that corresponds to S39 of SEQ ID NO: 16 is H;
(c) the amino acid that corresponds to A95 of SEQ ID NO: 16 is H;
(d) the amino acids that correspond to Y34 and S39 of SEQ ID NO: 16 are H;
(e) the amino acids that correspond to Y34 and A95 of SEQ ID NO: 16 are H;
(f) the amino acids that correspond to S39 and A95 of SEQ ID NO: 16 are H; or
(g) the amino acids that correspond to Y34, S39 and A95 of SEQ ID NO: 16 are H.

19. The engineered polypeptide of any one of claims 14-18, further comprising a PD-L1 surface interaction sequence with at least 5 amino acids, wherein the PD-L1 surface interaction sequence comprises two or more histidine residues.

20. The engineered polypeptide of claim 19, wherein the PD-L1 surface interaction sequence is at the N-terminus of the engineered polypeptide.

21. The engineered polypeptide of claim 19 or 20, wherein the PD-L1 surface interaction sequence is selected from the group consisting of: SHGHGGG, SHHGHGHGGGG, SHGHHGHGGGG and SHGHGHHGGGG.

22. The engineered polypeptide of any one of claims 19-21, wherein the PD-L1 surface interaction sequence has 5-15 amino acids.

23. The engineered polypeptide of any one of claims 19-22, wherein the PD-L1 surface interaction sequence interacts with E58/E60/D61 of PD-L1.

24. The engineered polypeptide of any one of claims 1-23, wherein the engineered polypeptide comprises an amino acid sequence that is at least 80%, 85%, 90%, or 95% identical to any one of SEQ ID NO: 1-16.

25. The engineered polypeptide of any one of claims 1-24, wherein the engineered polypeptide further comprises a CH2 domain and a CH3 domain.

26. The engineered polypeptide of any one of claims 1-25, wherein the engineered polypeptide further comprises a hinge region.

27. The engineered polypeptide of claim 25, wherein the CH2 domain is an IgG CH2 domain and the CH3 domain is an IgG CH3 domain.

28. The engineered polypeptide of any one of claims 1-27, wherein the engineered polypeptide comprises an amino acid sequence that is at least 80%, 85%, 90%, or 95% identical to any one of SEQ ID NO: 19-34.

29. A protein construct comprising the engineered polypeptide of any one of claims 1-28.

30. The protein construct of claim 29, comprising at least two engineered polypeptides.

31. The protein construct of claim 30, wherein the at least two engineered polypeptides are identical.

32. The protein construct of claim 30, wherein the at least two engineered polypeptides are different.

33. The protein construct of claim 29, further comprising an Fc region.

34. The protein construct of claim 33, wherein the Fc region is an IgG4 Fc region.

35. A protein construct comprising a first fusion polypeptide comprising the engineered polypeptide of any one of claims 1-28, a first CH2 domain, and a first CH3 domain; a second fusion polypeptide comprising a second CH2 domain, and a second CH3 domain, wherein the first fusion polypeptide and the second fusion polypeptide associate with each other, forming a dimer.

36. The protein construct of claim 35, wherein the second fusion polypeptide further comprises a second engineered polypeptide.

37. A pharmaceutical composition comprising the engineered polypeptide of any one of claims 1-28 or the protein construct of any one of claims 29-36; and a pharmaceutically acceptable carrier.

38. A nucleic acid encoding the engineered polypeptide of any one of claims 1-29 or the protein construct of any one of claims 29-36.

39. A vector comprising the nucleic acids of claim 38.

40. A cell comprising the nucleic acids of claim 38.

41. The cell of claim 40, wherein the cell is a CHO cell.

42. A method of producing an engineered polypeptide or a protein construct comprising the engineered polypeptide, the method comprising
(a) culturing the cell of claim 40 or 41 under conditions sufficient for the cell to produce the engineered polypeptide or the protein construct; and
(b) collecting the engineered polypeptide or the protein construct produced by the cell.

43. A method of treating a subject having cancer, the method comprising administering a therapeutically effective amount of a composition comprising the engineered polypeptide of any one of claims 1-28 or the protein construct of any one of claims 29-36, to the subject.

44. The method of claim 43, wherein the cancer cells express PD-L1.

45. The method of claim 43 or 44, wherein the cancer is melanoma, Hodgkin lymphoma, bladder cancer, kidney cancer, breast cancer, lung cancer (e.g., non-small cell lung cancer), head and neck squamous cell cancer, liver cancer, esophageal squamous cell cancer, colorectal cancer, cutaneous squamous cell carcinoma, or merkel cell carcinoma.

46. A method of decreasing the rate of tumor growth, the method comprising contacting a tumor cell with an effective amount of a composition comprising the engineered polypeptide of any one of claims 1-28 or the protein construct of any one of claims 29-36.

47. A method of killing a tumor cell, the method comprising contacting a tumor cell with an effective amount of a composition comprising the engineered polypeptide of any one of claims 1-28 or the protein construct of any one of claims 29-36.


SEQUENCE LISTING (40 sequences)

SEQ ID NO: 1 - protein, 136 aa, Synthetic construct
PTFSPALLVVTEGDNATFTCSFSNTSESFVLNWHRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLV

SEQ ID NO: 2 - protein, 136 aa, Synthetic construct
PTFSPALLVVTEGDNATFTCSFSNTSESFVLNWYRMSPHNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLV

SEQ ID NO: 3 - protein, 136 aa, Synthetic construct
PTFSPALLVVTEGDNATFTCSFSNTSESFVLNWYRMSPSNQTDKLAAFPEDRSQPGQDCRFRVTQLPNGRDFHMSVVRARRNDSGTYLCGAISLHPKAQIKESLRAELRVTERRAEVPTAHPSPSPRPAGQFQTLV

SEQ ID NO: 4 - protein, 136 aa, Synthetic construct
PTFSPALLVVTEGDNATFTCSFSNTSESFVLNWHRMSPHNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLV

SEQ ID NO: 5 - protein, 136 aa, Synthetic construct
PTFSPALLVVTEGDNATFTCSFSNTSESFVLNWHRMSPHNQTDKLAAFPEDRSQPGQDCRFRVTQLPNGRDFHMSVVRARRNDSGTYLCGAISLHPKAQIKESLRAELRVTERRAEVPTAHPSPSPRPAGQFQTLV

SEQ ID NO: 6 - protein, 143 aa, Synthetic construct
SHGHGGGPTFSPALLVVTEGDNATFTCSFSNTSESFVLNWYRMSPHNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLV

SEQ ID NO: 7 - protein, 143 aa, Synthetic construct
SHGHGGGPTFSPALLVVTEGDNATFTCSFSNTSESFVLNWHRMSPHNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLV

SEQ ID NO: 8 - protein, 143 aa, Synthetic construct
SHGHGGGPTFSPALLVVTEGDNATFTCSFSNTSESFVLNWHRMSPHNQTDKLAAFPEDRSQPGQDCRFRVTQLPNGRDFHMSVVRARRNDSGTYLCGAISLHPKAQIKESLRAELRVTERRAEVPTAHPSPSPRPAGQFQTLV

SEQ ID NO: 9 - protein, 147 aa, Synthetic construct
SHHGHGHGGGGPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLV

SEQ ID NO: 10 - protein, 147 aa, Synthetic construct
SHGHHGHGGGGPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLV

SEQ ID NO: 11 - protein, 147 aa, Synthetic construct
SHGHGHHGGGGPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLV

SEQ ID NO: 12 - protein, 147 aa, Synthetic construct
SHHGHGHGGGGPTFSPALLVVTEGDNATFTCSFSNTSESFVLNWYRMSPHNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLV

SEQ ID NO: 13 - protein, 147 aa, Synthetic construct
SHGHHGHGGGGPTFSPALLVVTEGDNATFTCSFSNTSESFVLNWYRMSPHNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLV

SEQ ID NO: 14 - protein, 147 aa, Synthetic construct
SHGHGHHGGGGPTFSPALLVVTEGDNATFTCSFSNTSESFVLNWYRMSPHNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLV

SEQ ID NO: 15 - protein, 145 aa, Homo sapiens
DSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLV

SEQ ID NO: 16 - protein, 136 aa, Homo sapiens
PTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLV

SEQ ID NO: 17 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 18 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 19 - protein, 374 aa, Synthetic construct
DSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 20 - protein, 365 aa, Synthetic construct
PTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 365 aa, Synthetic construct
PTFSPALLVVTEGDNATFTCSFSNTSESFVLNWHRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - protein, 365 aa, Synthetic construct
PTFSPALLVVTEGDNATFTCSFSNTSESFVLNWYRMSPHNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 23 - protein, 365 aa, Synthetic construct
PTFSPALLVVTEGDNATFTCSFSNTSESFVLNWYRMSPSNQTDKLAAFPEDRSQPGQDCRFRVTQLPNGRDFHMSVVRARRNDSGTYLCGAISLHPKAQIKESLRAELRVTERRAEVPTAHPSPSPRPAGQFQTLVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 24 - protein, 365 aa, Synthetic construct
PTFSPALLVVTEGDNATFTCSFSNTSESFVLNWHRMSPHNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 25 - protein, 365 aa, Synthetic construct
PTFSPALLVVTEGDNATFTCSFSNTSESFVLNWHRMSPHNQTDKLAAFPEDRSQPGQDCRFRVTQLPNGRDFHMSVVRARRNDSGTYLCGAISLHPKAQIKESLRAELRVTERRAEVPTAHPSPSPRPAGQFQTLVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 26 - protein, 372 aa, Synthetic construct
SHGHGGGPTFSPALLVVTEGDNATFTCSFSNTSESFVLNWYRMSPHNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - protein, 372 aa, Synthetic construct
SHGHGGGPTFSPALLVVTEGDNATFTCSFSNTSESFVLNWHRMSPHNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 28 - protein, 372 aa, Synthetic construct
SHGHGGGPTFSPALLVVTEGDNATFTCSFSNTSESFVLNWHRMSPHNQTDKLAAFPEDRSQPGQDCRFRVTQLPNGRDFHMSVVRARRNDSGTYLCGAISLHPKAQIKESLRAELRVTERRAEVPTAHPSPSPRPAGQFQTLVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 29 - protein, 376 aa, Synthetic construct
SHHGHGHGGGGPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 30 - protein, 376 aa, Synthetic construct
SHGHHGHGGGGPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 31 - protein, 376 aa, Synthetic construct
SHGHGHHGGGGPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 32 - protein, 376 aa, Synthetic construct
SHHGHGHGGGGPTFSPALLVVTEGDNATFTCSFSNTSESFVLNWYRMSPHNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 33 - protein, 376 aa, Synthetic construct
SHGHHGHGGGGPTFSPALLVVTEGDNATFTCSFSNTSESFVLNWYRMSPHNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 34 - protein, 376 aa, Synthetic construct
SHGHGHHGGGGPTFSPALLVVTEGDNATFTCSFSNTSESFVLNWYRMSPHNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 35 - protein, 229 aa, Synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 36 - protein, 7 aa, Synthetic construct
SHGHGGG

SEQ ID NO: 37 - protein, 11 aa, Synthetic construct
SHHGHGHGGGG

SEQ ID NO: 38 - protein, 11 aa, Synthetic construct
SHGHHGHGGGG

SEQ ID NO: 39 - protein, 11 aa, Synthetic construct
SHGHGHHGGGG

SEQ ID NO: 40 - protein, 9 aa, Synthetic construct
DSPDRPWNP